SEQUENCE LISTING (4 sequences)

SEQ ID NO: 1 - protein, 427 aa, Homo sapiens
  human interferon regulatory factor 3 (IRF3) cDNA
MGTPKPRILPWLVSQLDLGQLEGVAWVNKSRTRFRIPWKHGLRQDAQQEDFGIFQAWAEATGAYVPGRDKPDLPTWKRNFRSALNRKEGLRLAEDRSKDPHDPHKIYEFVNSGVGDFSQPDTSPDTNGGGSTSDTQEDILDELLGNMVLAPLPDPGPPSLAVAPEPCPQPLRSPSLDNPTPFPNLGPSENPLKRLLVPGEEWEFEVTAFYRGRQVFQQTISCPEGLRLVGSEVGDRTLPGWPVTLPDPGMSLTDRGVMSYVRHVLSCLGGGLALWRAGQWLWAQRLGHCHTYWAVSEELLPNSGHGPDGEVPKDKEGGVFDLGPFIVDLITFTEGSGRSPRYALWFCVGESWPQDQPWTKRLVMVKVVPTCLRALVEMARVGGASSLENTVDLHISNSHPLSLTSDQYKAYLQDLVEGMDFQGPGES

SEQ ID NO: 2 - DNA, 1648 bp, Homo sapiens
  human interferon regulatory factor 3 (IRF3)
cgtagaaccagataggggcgggaacagcccagcgggccgtcccatcggcttttgggtctgttacccaaagaatgataaagttggttttatttcaagaagtcgatcgaaaagaaagccccagcgctctagagctcagctgacgggaaagggggtgcgcagcctcgagtttgagagctacccggagccccaagacaggggggggttccagctgcccgcacgccccgaccttccatcgtaggccggaccatgggaaccccaaagccacggatcctgccctggctggtgtcgcagctggacctggggcaactggagggcgtggcctgggtgaacaagagccgcacgcgcttccgcatcccttggaagcacggcctacggcaggatgcacagcaggaggatttcggaatcttccaggcctgggccgaggccactggtgcatatgttcccgggagggataagccagacctgccaacctggaagaggaatttccgctctgccctcaaccgcaaagaagggttgcgtttagcagaggaccggagcaaggaccctcacgacccacataaaatctacgagtttgtgaactcaggagttggggacttttcccagccagacacctctccggacaccaatggtggaggcagtacttctgatacccaggaagacattctggatgagttactgggtaacatggtgttggccccactcccagatccgggacccccaagcctggctgtagcccctgagccctgccctcagcccctgcggagccccagcttggacaatcccactcccttcccaaacctggggccctctgagaacccactgaagcggctgttggtgccgggggaagagtgggagttcgaggtgacagccttctaccggggccgccaagtcttccagcagaccatctcctgcccggagggcctgcggctggtggggtccgaagtgggagacaggacgctgcctggatggccagtcacactgccagaccctggcatgtccctgacagacaggggagtgatgagctacgtgaggcatgtgctgagctgcctgggtgggggactggctctctggcgggccgggcagtggctctgggcccagcggctggggcactgccacacatactgggcagtgagcgaggagctgctccccaacagcgggcatgggcctgatggcgaggtccccaaggacaaggaaggaggcgtgtttgacctggggcccttcattgtagatctgattaccttcacggaaggaagcggacgctcaccacgctatgccctctggttctgtgtgggggagtcatggccccaggaccagccgtggaccaagaggctcgtgatggtcaaggttgtgcccacgtgcctcagggccttggtagaaatggcccgggtagggggtgcctcctccctggagaatactgtggacctgcacatttccaacagccacccactctccctcacctccgaccagtacaaggcctacctgcaggacttggtggagggcatggatttccagggccctggggagagctgagccctcgctcctcatggtgtgcctccaacccccctgttccccaccacctcaaccaataaactggttcctgctatgaaaaaaaaaaaaaaaaaaaaaaaaaaaaaaaaaaaaaaaaaaa

SEQ ID NO: 3 - DNA, 19 bp, Artificial Sequence
  Description of Artificial Sequence transcriptional repressor Hes1 siRNA target sequence
cgacaccggacaaaccaaa

SEQ ID NO: 4 - DNA, 17 bp, Artificial Sequence
  Description of Artificial Sequenceretinoid X receptor alpha (RXRalpha) siRNA target sequence
aagcacaggaggacagc

(19) United States
(12) Patent Application Publication
(10) Pub. No.: US 2009/0181910 A1
(43) Pub. Date: Jul. 16, 2009

(54) METHOD OF PREVENTION AND ALLEVIATION OF TOXICITY BY MODULATION OF IRF3

(75) Inventors: Edward K. Chow, Rancho Palos Verdes, CA (US); Genhong Cheng, Calabasas, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA (US)

(21) Appl. No.: 11/867,589

(22) Filed: Oct. 4, 2007

Related U.S. Application Data
(60) Provisional application No. 60/849,899, filed on Oct. 6, 2006.

(51) Int. Cl. A61K 31/713; A61K 31/16; A61K 31/715; A61K 31/437; A61K 31/195; A61P 3/00

(52) U.S. Cl. 514/44; 514/630; 514/54; 514/293; 514/562

(57) ABSTRACT

The invention provides compounds, compositions, animal models, drug screening methods, pharmaceutical compositions, and methods of treatment which relate to the modulation of the metabolism of xenobiotic compounds by administering agents which act on IRF3 or an IRF3 control pathway to modulate the activity, expression, or levels of cytochrome P450 enzymes involved in the metabolism of xenobiotic compounds in a subject.

### CROSS-REFERENCES TO RELATED APPLICATIONS

This application claims priority benefit of U.S. Provisional Patent Application Ser. No. 60/849,899, filed on Oct. 6, 2006, which is incorporated by reference in its entirety for all purposes.

### STATEMENT AS TO RIGHTS TO INVENTIONS MADE UNDER FEDERALLY SPONSORED RESEARCH AND DEVELOPMENT THIS WORK WAS SUPPORTED IN PART BY NATIONAL INSTITUTES OF HEALTH RESEARCH GRANTS R01 CA87924, R01 AI056154 AND HL30568.

Reference to a “Sequence Listing,” a table, or a computer program listing appendix submitted on a compact disk.

### BACKGROUND OF THE INVENTION

There is growing evidence that viral infections contribute to the induction or progression of metabolic diseases, potentially through inflammation and other unknown mechanisms. Viral infections have been linked to defects in cholesterol metabolism (Alber et al., Circulation, 102:779-785 (2000)), such as atherosclerosis, and liver metabolism of drugs as in Reye's Syndrome (Ruben et al., Am J Public Health, 66:1096-1098 (1976)), as well as bone metabolism defects, skin eruptions and diabetes (Mondy, K. and Tebas, P., Clin Infect Dis, 36:S101-105 (2003); Shaker et al., J Clin Endocrinol Metab, 83:93-98 (1998); Ratziu et al., Aliment Pharmacol Ther, 22 Suppl 2:56-60 (2005); Michitaka et al., Intern Med, 43:696-699)). There is also evidence that maternal viral infections can lead to the maternal immune system affecting embryonic development, as seen in TORCH infections (Shi et al., Int J Dev Neurosci, 23:299-305 (2005)).

A common mechanism in the development of metabolic disorders is the alteration of gene expression controlled by nuclear hormone receptors. Members of this family function as transcriptional regulators of metabolic pathways in multiple cell types. Retinoic X receptors (RXRs) play a uniquely important role in metabolism due to their ability to form heterodimers with many different nuclear receptors, including PPARs LXR, FXR, VDR, TR, PXR and CAR (Carlberg et al, Nature, 361:657-660 (1993); Leid et al., Cell 68:377-395 (1992)). Thus, any signal that alters RXR function or expression has the potential to impact multiple different metabolic programs. A range of intermediates or end products of metabolic pathways, including bile acids, fatty acids, oxysterols and steroids have been shown to regulate gene expression through direct binding to RXR heterodimeric receptors (Tontonoz et al., Cell, 79:1147-1156 (1994); Tontonoz et al., Genes Dev, 8:1224-1234 (1994); Willy et al., Genes Dev, 9:1033-1045 (1995); Xie et al., Proc Natl Acad Sci, 98:3375-3380 (2001); Sucov et al., Genes Dev, 8:1007-1018 (1994); Janowski et al., Nature, 383:728-731 (1996); Kliewer et al., Cell, 92:73-82 (1998); Sakashita et al., Blood, 81:1009-1016 (1993); Wu et al., Mol Pharmacol, 65:550-557 (2004); Makishima et al., Science, 284:1362-1365 (1999); Makishima et al., Science 296:1313-1316 (2002); Imai et al., Proc Natl Acad Sci USA, 98:224-228 (2001)). Two different RXR heterodimer partners, CAR and PXR, are activated by xenobiotics and participate in hepatic detoxification pathways. Studies using knockout mice have confirmed that these proteins are essential for proper steroid, drug and xenobiotic metabolism (Xie et al., Proc Natl Acad Sci, 98:3375-3380 (2001); Wu et al., Mol Pharmacol, 65:550-557 (2004); Makishima et al., Science, 284:1362-1365 (1999); Makishima et al., Science 296:1313-1316 (2002); Staudinger et al., Proc Natl Acad Sci USA, 98:3369-3374 (2001)). Challenging these mice with xenobiotics or toxic bile acids leads to fatty degeneration, acute-liver failure and death.

Previous work has pointed to the existence of crosstalk between nuclear receptor signaling and the innate immune response. Induction of acute phase response by treating mice with LPS has been associated with the down regulation of certain nuclear receptors in the liver, including RXR (Beigneux et al., J Biol Chem, 275:16390-16399 (2000); Beigneux et al., Biochem Biophys Res Commun, 293:145-149 (2002); Kim et al., J Biol Chem, 278:8988-8995 (2003)). Recently the induction of an anti-viral immune response in macrophages has been shown to inhibit LXR/RXR function and cholesterol efflux, suggesting a possible mechanism for viral-induced foam cell formation in atherosclerosis (Castrillo et al., Mol Cell, 12:805-816 (2003)). Although the precise mechanisms whereby bacterial or viral infections inhibit nuclear receptor function are unknown, studies on LXR have implicated interferon regulatory factor 3 (IRF3) (Castrillo et al., Mol Cell, 12:805-816 (2003)).

IRF3 is a transcription factor shared by both LPS signaling and the anti-viral immune response. Upon viral infection or stimulation with toll-like receptor agonists such as polyI:C or LPS, IRF3 is phosphorylated by serine/threonine kinase, TANK binding kinase 1 (TBK1) or Inducible IκB kinase (IKKi) (Perry et al., J Exp Med, 199:1651-1658 (2004)). In addition to being activated by TLR-TRIF-dependent pathways (Yamamoto et al., Science, 301:640-643 (2003)), intracellular receptors such as RIG-I are capable of activating IRF3 upon recognition of polyI:C and RNA viruses (Li et al., J Biol Chem, 280:16739-16747 (2005); Yoneyama et al., Nat Immunol, 5:730-737 (2004)). Following activation, IRF3 promotes transcription of Type I IFN genes together with other transcription factors such as NF-κB and AP-1 (Perry et al., J Exp Med, 199:1651-1658 (2004); Li et al., J Biol Chem, 280:16739-16747 (2005); Jiang et al., Proc Natl Acad Sci USA, 101:3533-3538 (2004)). Although IRF3's role in Type I IFN induction is well established, there is emerging data demonstrating that IRF3 also functions as a coactivator of NF-κB in the LPS response (Leung et al., Cell 118:453-464 (2004); Ogawa et al., Cell, 122:707 72 (2004)). Mechanisms whereby IRF3 might function to repress target gene expression, however, have not been elucidated.

Acetaminophen (APAP) is the leading cause of acute liver failure in the United States. APAP hepatotoxicity occurs when a more toxic intermediate, N-acetyl-p-benzoquinone-imine (NAPQI), is made that can be processed by glutathione S-transferase (GST) enzymes. Biotransformation process by which NAPQI is made occurs through cytochrome P450 family members (CYPs) In addition to being caused by overdose from incorrect usage of APAP, hepatotoxicity can also occur through combinatorial ingestion of APAP and CYP inducing drugs and compounds like ethanol. Here, we describe a novel method for prevention of such mechanisms of APAP hepatotoxicity through the activation of IRF3 and other factors by polyI:C. PolyI:C transcriptionally represses RXRα and RXRα target CYPs through activation of IRF3 and other factors. This repression of RXRα and CYPs effectively prevents APAP hepatotoxicity and overdose, providing a novel method for preventing APAP hepatotoxicity.

Acetaminophen (APAP) overdose accounts for 49% of all acute liver failure cases (Lazerow et al., Curr Opin Gastroenterol 21, 283-292. (2005)). Furthermore, 20% of idiopathic liver failure cases had elevated APAP levels in serum (Lazerow et al., Curr Opin Gastroenterol 21, 283-292. (2005)). APAP hepatotoxicity occurs due to saturation of the metabolic pathway, resulting in increased toxic intermediate metabolites. During normal metabolism of APAP, bioactivation by cytochrome P450 family members, Cyp3A11, Cyp1A2 and Cyp2E1, transforms APAP into N-acetyl-p-benzoquinone-imine (NAPQI) (Dahlin et al., Proc Natl Acad Sci USA, 81, 1327-1331 (1984); Gonzalez, F. J., and Kimura, S., Arch Biochem Biophys 409, 153-158 (2003); Guo et al., Toxicol Sci 82, 374-380 (2004)). NAPQI is a highly reactive toxic intermediate that normally is conjugated with glutathione (GSH) by glutathione S-transferase (GST) enzymes creating a more hydrophilic form that is easily excreted (Mitchell et al., J Pharmacol Exp Ther 187, 211-217 (1973)). When APAP's metabolic pathway becomes saturated, NAPQI forms faster than it can be GSH conjugated and excreted. NAPQI is then capable of covalently binding to nucleophilic cellular macromolecules causing cell death and toxicity (Jollow et al., Pharmacology 12, 251-271 (1974)).

Cytochrome P450 family members (CYPs) play an important role in the development of APAP and chemical induced hepatotoxicity generally. Gene expression of many of these family members that are involved in APAP metabolism is controlled by nuclear receptors. Nuclear receptors are transcription factors that control a number of biological processes ranging from metabolism to development (Szanto et al., Cell Death Differ 11 Suppl 2, S126-143A (2004)). One key nuclear receptor that regulates the expression of CYPs is Retinoid X Receptor (RXRα) (Wu et al., Mol Pharmacol 65, 550-557 (2004)). RXRα is required for high expression of Cyp3A11 and Cyp1A2 and is critical to the development of APAP hepatotoxicity (Wu et al., Mol Pharmacol 65, 550-557 (2004)). RXRα primarily functions as a critical heterodimeric partner with other nuclear receptors to recruit transcriptional activators and transcriptional machinery (Dilworth et al., Mol Cell, 6, 1049-1058 (2000)). These other nuclear receptors that have been implicated in APAP induced hepatotoxicity because of their role in the expression of CYPs include pregnane X receptor (PXR)/steroid xenobiotic receptor (SXR) (Guo et al., Toxicol Sci 82, 374-380 (2004)) and constitutive androstane receptor (CAR) (Zhang et al., Science 298, 422-424 (2002)). Activation of these nuclear receptors by xenobiotics and drugs increases expression of CYPs. It is this increased CYP expression that promotes APAP-induced hepatic injury. Other substances that increase CYP expression and have been implicated with increased sensitivity to APAP hepatotoxicity include ethanol (McClain et al., Jama 244, 251-253 (1980)).

Recently, we and other labs have identified inhibitory crosstalk between nuclear receptors and anti-viral immune responses (Castrillo et al., Mol Cell 12, 805-816 (2003)). Viral particles, such as dsRNA, can activate an anti-viral immune response that activates transcription factors NF-κB and IRF3 through a variety of receptors, including Toll-like receptor 3 (TLR3) which activated these transcription factors through TRIF (Doyle et al., Immunity 17, 251-263 (2002); Jiang et al., Proc Natl Acad Sci USA 101, 3533-3538 (2004)).

APAP induced hepatotoxicity is a dangerous disease that results from the production of the toxic intermediate, NAPQI, than its safer GSH conjugated form. APAP hepatotoxicity is dependent on CYPs which are regulated by nuclear receptors such as RXRα. Cyp3A11 and Cyp1A2 expression involves RXRα and other nuclear receptors. These CYPs participate in the biotransformation of APAP into NAPQI. Hepatocyte-specific RXRα deficient mice exhibit lower expression of Cyp3A11 and Cyp1A2 and are highly resistant to APAP induced hepatotoxicity (Dai et al, Exp Mol Pathol 75, 194-200 (2003); Wu et al., Mol Pharmacol 65, 550-557 (2004)). Similar results occur in mice deficient in RXRα's heterodimeric partners, PXR and CAR (Guo et al., Toxicol Sci 82, 374-380 (2004); Zhang et al., Science 298, 422-424 (2002)).

Current treatment for APAP is intravenous or oral N-acetylcysteine (NAC) therapy. NAC treatment must occur within the first 10 hours of APAP ingestion in order to be effective (Tsai et al., Clin Ther 27, 336-341 (2005)). NAC serves as an antidote to APAP overdose by increasing glutathione (GSH) levels, as well as binding to NAPQI and serving as an antioxidant (Rafeiro et al., Toxicology 93, 209-224 (1994)). This method of protecting against APAP overdose requires the cases of overdose to be identified within the first 10 hours of APAP ingestion. It does not protect against APAP overdose at the most critical time when APAP is actually being ingested and transformed into the toxic intermediate, NAPQI.

Accordingly, there is a need for additional therapies for treating subjects who have been exposed to compounds, such as acetaminophen, which are metabolized by enzymes of the cytochrome P450 enzyme family. This invention meets these and other needs by providing methods and compositions which act by modulating IRF3 to influence the expression and tissue levels of enzymes of the Cytochrome P450 family. These IRF3 modulators find particular application in treating subjects needing protection from toxicity associated with exposure to, or administration of, xenobiotic compounds.

### BRIEF SUMMARY OF THE INVENTION

This invention relates to the finding that tissue levels or expression of cytochrome P450 enzymes can be modulated by administering to a mammalian subject an agent which modulates IRF3 expression, levels, or activity in the tissue.

In a first aspect, this invention provides a method for reducing tissue levels or expression of cytochrome P450 enzymes by administering to a mammalian subject an IRF3 modulator or a modulator of one or more members of the IRF3 pathway set forth in FIGS. 5 and 13 which influence Cytochrome P450 enzyme activity, levels or expression in a tissue. In some embodiments, the modulator is directly or indirectly an activator or agonist or inducing agent for IRF3. In some embodiments, the subject has been exposed to or administered a compound, is suspected of having been exposed or administered to a compound (e.g., a xenobiotic compound, drug, or naturally occurring toxin) or is expected to or has a substantial likelihood of being exposed to or administered to a compound whose toxicity is increased by the activity of a Cytochrome P450 enzyme. In such embodiments, the subject is in need of a reduced Cytochrome P450 levels or activity in order to reduce the toxicity of the compound which is metabolized to a more toxic compound by the action of a cytochrome P450 enzyme whose expression or activity or levels is reduced by the administration of an IRF3 modulator. In some further embodiments, the tissue is the liver, lung, intestines, or kidney. In some embodiments, the agent is a toll-like receptor agonist. In some embodiments, the hepatotoxicity of the xenobiotic compound is reduced. In one embodiment, the compound is acetaminophen and the hepatotoxicity of acetaminophen is reduced by administration of the IRF3 activator or agonist. In still other embodiments of any of the above, the modulator is polyI:C or LPS. In some further embodiments, the xenobiotic compound (e.g., acetaminophen) is co-administered with the polyI:C. In still further embodiments, the polyI:C is administered after the xenobiotic compound (e.g., acetaminophen). In additional embodiments, the subject is a human who is suspected of having or has ingested an overdose of acetaminophen or another xenobiotic compound that can be metabolized to a toxic metabolite by a cytochrome P450 enzyme whose activity, expression, or levels is modulated or reduced by administration of an IRF3 activator to the subject.

In some further embodiments of the above, the xenobiotic compound is a halogenated compound. In additional embodiments, the xenobiotic compound is a procarcinogen and the metabolite is a carcinogen. In some embodiments, the compound or metatabolite is a hepatocarcinogen, a lung carcinogen, a kidney carcinogen, or an carcinogen of the gastrointestinal tract which is metabolized via a cytochrome P450 enzyme in the corresponding tissue. In other embodiments, the xenobiotic compound is metabolized by a cytochrome P450 enzyme to form a reactive intermediate which is capable of covalently reacting with tissue macromolecules. In other embodiments, the metabolite is a free radical or can become converted to a free radical in the body.

In further embodiments of any of the above, the cytochrome P450 enzyme is Cytochrome P450 3A11 or Cytochrome P450 1A2. In still other embodiments, the cytochrome P450 enzyme comprises a Cytochrome P450 isoform selected from Cytochrome P450 1A2, Cytochrome P450 2B6, Cytochrome P450 2C19, Cytochrome P450 2C9, Cytochrome P450 2D6, Cytochrome P450 2E1, and Cytochrome P450 3A 4, 5, or 7.

In some embodiments of any of the above the IRF3 modulator is polyI:C.

In some embodiments of any of the above, the mammalian subject is a human, a primate, a cat, dog, rodent, lagamorph, rat mouse, guinea pig, hamster. In some embodiments, the subject was exposed to an inducer of the Cytochrome P450 enzyme. In some embodiments, the effects of the IRF3 modulator and/or xenobiotic compound on an affected organ are measured by organ function tests or histocytochemical/morphology studies of tissue from the organ of interest. In some embodiments, the effects of the IRF3 modulator in protecting the liver from the toxicity of the compound are monitored by using liver function test, serum ALT levels, AST levels, bilirubin levels, alkaline phosphatase levels, or albumin levels or histocytochemistry/morphology studies of liver tissue from the subject. In some embodiments, the subject has a condition which increases their susceptibility to the xenobiotic agent (e.g., depleted glutathione stores, increased induction of members of the Cytochrome P450 enzyme system involved in the metabolism of the agent, malnutrition). The IRF3 modulator may be administered by any route, including the oral, subcutaneous, intramuscular, intraperitoneal, and intravenous routes.

In this aspect, the invention also provides methods of modulating the metabolism of a compound by Cytochrome P450 enzyme system in a mammal (e.g., human) exposed to the compound by administering an effective amount of polyI:C to a patient before, during or after the exposure to the compound. In some embodiments, the compound is one whose major route of the metabolism or disposition in the subject is by the Cytochrome P450 enzyme system. In further embodiments, this metabolism or disposition mediates a toxicity of the compound. In any embodiments of the above, the toxic compound can be a drug and the exposure can be by administration of the drug to the subject. In an exemplary embodiment, the compound is acetaminophen. In some embodiments, the subject is an adult human who was administered or has ingested an overdose of acetaminophen (e.g., more than 3, 5, or 7 times the recommended therapeutic dosage for a preparation; or ingested more than 8 g/day, 10 g.day, or 20 g in one day; or ingested or was administered an overdose over several successive days). In another embodiment, the modulation provides a means of preventing or reducing the induction of a Cytochrome P450 enzyme in a subject exposed to a substance capable of inducing the enzyme, said method comprising administering an effective amount of polyI:C to the subject.

In another aspect, the invention provides a pharmaceutical composition comprising a first compound which is a drug substrate for a Cytochrome 450 enzyme system and a second agent which is a modulator of IRF3 or members of the IRF3 activation pathways set forth in FIGS. 5 and 13 which influence cytochrome P450 activity, expression or levels (e.g., polyI:C). In some embodiments, the compound is a drug (e.g., acetaminophen) which is metabolized to a toxic compound by the action of the Cytochrome 450 enzyme system. In another embodiment, the composition comprises a first agent which induces a Cytochrome P450 enzyme and a second agent which is polyI:C. In further embodiments, the cytochrome P450 enzyme is Cytochrome P450 3A11 or Cytochrome P450 1A2. In still other embodiments, the cytochrome P450 enzyme comprises a Cytochrome P450 isoform selected from Cytochrome P450 1A2, Cytochrome P450 2B6, Cytochrome P450 2C19, Cytochrome P450 2C9, Cytochrome P450 2D6, Cytochrome P450 2E1, and Cytochrome P450 3A 4, 5, or 7.

In some embodiments, the invention provides a pharmaceutical composition comprising polyI:C and acetaminophen and optionally N-acetylcysteine. The composition may be formulated for any route of administration, including the oral, rectal, subcutaneous, intramuscular, intraperitoneal, and intravenous routes.

In another aspect, the invention relates to the discovery of the critical role of IRF3-dependent RXRα repression in the xenobiotic hepatotoxicity associated with viral infections. In some embodiments in this aspect, the invention provides methods of screening or identifying drugs, natural substances, and xenobiotic compounds which may have an infection-mediated or -augmented toxicity. For instance, the xenobiotic compound (e.g., environmental or industrial chemical, or drug (e.g., aspirin)) which is normally detoxified by metabolism to a non-toxic compound by the action of a cytochrome P450 enzyme is more toxic when the activity of this detoxification pathway is reduced by infection or an agent which modulates IRF3 activity and consequently the levels, expression, and/or activity of the Cytochrome P450 enzyme mediating metabolism of the drug or compound. The effect may be assessed by monitoring liver function as described above in animals. In some embodiments, a drug or xenobiotic compound is identified as being a candidate for infection-mediated or -augmented toxicity by determining the effect of an infection or infection-mimicking agent (e.g., LPS, poly I:C) on expression of a cytochrome P450 enzyme involved in the detoxifying metabolism of the compound. Alternatively, the toxicity of a xenobiotic compound in a test animal which has been infected with a pathogen of interest or given an infection-mimicking agent can be compared to a test animal not so treated (e.g., a control). (e.g., LPS, poly I:C). In some embodiments, the ability of a substance to cause hepato-toxicity in an infected patient is assessed by identifying whether the expression of a cytochrome P450 enzyme known to be involved or shown to be involved in the detoxifying metabolism of the compound is altered during infection or by administration of an IRF3 activator. In some embodiments, the invention provides a method for protecting an infected subject from a toxicity associated with exposure to the xenobiotic wherein the infection is a risk factor for the toxicity by administering an agent which inhibits IRF3 or increases the expression of RXRα.

In another aspect, the invention provides animal models for studying the effects of infectious agents on the toxicity of compounds, including xenobiotics. In one embodiment, the invention provides animal models for studying the effects of infectious agents on the toxicity of compounds by comparing the toxicity of a compound between infected or uninfected animal administered the compound of interest. In another embodiment, the invention provides animal models for studying the effects of infectious agents on the toxicity of compounds by comparing the toxicity of a compound between animals given an agent which mimics an infection (e.g., poly I:C, LPS, endotoxin) and animals not given the agent. In some embodiments, the toxic compound is aspirin. In some embodiments, the toxicity is hepatotoxicity, cardiotoxicity, renal toxicity, pancreatic toxicity, or a CNS and/or PNS toxicity. In some embodiments, the toxicity is a metabolic disorder such as type I or type II diabetes, insulin resistance, hyperlipidemia, hypercholesterolemia. In some embodiments, the test species is a mouse and the compound is administered to both control mice and infected mice or mice administered an IRF3 modulatory compound to determine the effect of the modulator on the toxicity of the compound.

### BRIEF DESCRIPTION OF THE DRAWINGS

FIG. 1: PolyI:C repression of RXRα and cytochrome P450 family members involves IRF3. Wildtype or IRF3−/− mice (n=4) fasted and were treated with 0.1% NaCl or polyI:C (100 μg) intravenous (i.v.) 24 hours prior to treatment with APAP (350 mg/kg) by intraperitoneal (i.p.) injection. 6 hours post-APAP treatment liver samples were isolated. Liver RNA analyzed by Q-PCR for (A) RXRα (B) Cyp1A2 and (C) Cyp3A11.

FIG. 2: PolyI:C prevents serum ALT induction by APAP with or without cytochrome P450 inducers. (A) Wildtype or IRF3−/− mice (n=4) fasted and were treated with 0.1% NaCl or polyI:C (100 μg) intravenous (i.v.) 24 hours prior to treatment with APAP (350 mg/kg) by intraperitoneal (i.p.) injection. 6 hours post-APAP treatment, serum samples were isolated and analyzed for serum ALT levels (TECO Diagnostic). (B) Wildtype mice (n=4) fasted and were treated with 0.1% NaCl or polyI:C (100 μg) intravenous (i.v.) and PCN (75 mg/kg) intraperitoneal (i.p.) 24 hours prior to treatment with APAP (175 mg/kg) by intraperitoneal (i.p.) injection. 6 hours post-APAP treatment, serum samples were isolated and analyzed for serum ALT levels (TECO Diagnostic). (C) Wildtype mice (n=4) were given 20% EtOH ad libidum for 5 days. 0.1% NaCl or polyI:C treatment (i.v.) was done on Day 3 and Day 5. Mice fasted 24 hours prior to APAP treatment (175 mg/kg) on Day 6. 6 hours post-APAP treatment, serum samples were isolated and analyzed for serum ALT levels (TECO Diagnostic).

FIG. 3: Histological Analysis of polyI:C inhibition of APAP hepatotoxicity. (A) 6 hours post-APAP treatment, liver samples were isolated and formalin fixed. Samples were stained with H&E. (B) Mice were treated as described in FIG. 2 B, C. Liver samples were formalin fixed and stained with H&E.

FIG. 4: Percentage survival following APAP treatment with or without polyI:C. (A) Wildtype (n=8) fasted and were treated with 0.1% NaCl or polyI:C (100 μg) intravenous (i.v.) 24 hours prior to treatment with APAP (600 mg/kg) by intraperitoneal (i.p.) injection. (B) Wildtype and IRF3−/− (n=6-8) fasted and were treated with 0.1% NaCl or polyI:C (100 μg) intravenous (i.v.) 24 hours prior to treatment with APAP (600 mg/kg) by intraperitoneal (i.p.) injection. (C) Wildtype mice (n=6-8) fasted and were treated with 0.1% NaCl or polyI:C (100 μg) intravenous (i.v.) and PCN (75 mg/kg) intraperitoneal (i.p.) 24 hours prior to treatment with APAP (175 mg/kg) by intraperitoneal (i.p.) injection.

FIG. 5: Model of polyI:C protection against APAP hepatotoxicity. Upon entering the liver, APAP is biotransformed into the toxic intermediate NAPQI by Cytochrome P450 family members (CYPs), Cyp3A11 and Cyp1A2. Increased formation of NAPQI by these CYPs results in cell injury and hepatotoxicity. These CYPs are target genes of RXRα and its heterodimeric nuclear receptor (NR) partners. PolyI:C treatment activates signaling cascades such as TLR3-TRIF-IRF3 that can repress RXRα and RXRα target genes. Repression of RXRα and CYPs by polyI:C limits the rate at which NAPQI is formed, preventing APAP hepatotoxicity.

FIG. 6: Viral infections negatively regulate in vivo RXR heterodimer target genes and liver metabolism. a,b Wildtype (n=4) were treated with 0.1% NaCl or VSV (2.5e7 pfu) intravenous (i.v.) on Day 1 with or without Vehicle (1% DMSO, corn oil), Pregnenolone-16alpha-carbonitrile (PCN) (75 mg/kg) by gavage for 4 days. Liver RNA was analyzed by Q-PCR. c, Wildtype (n=4) were treated with 0.1% NaCl or VSV (2.5e7 pfu) intravenous (i.v.) on Day 1 and Day 3 and Vehicle (1% DMSO, corn oil), PCN (75 mg/kg) by gavage and/or LCA (0.25 mg/kg) intraperitoneal (i.p.) for 4 days. Serum was collected and analyzed for serum alanine aminotransferase (ALT) as described in Materials and Methods. *P≦0.001 d, Representative Oil Red 0 staining of livers isolated following treatment in c.

FIG. 7: PolyI:C negatively regulated in vivo RXR heterodimer target genes and liver metabolism. a, Wildtype or IRF3^{-/-} mice (n=4) were treated with 0.1% NaCl or polyI:C (150 μg) intravenous (i.v.) on Day 1 and Day 3 with or without Vehicle (1% DMSO, corn oil), or PCN (75 mg/kg) by gavage for 4 days. Liver RNA analyzed by Q-PCR. b, Representative anti-RXRα and anti-USF2 Western Blot of wildtype livers after treatment with 0.1% NaCl or polyI:C (150 kg) intravenous (i.v.) on Day 1 and Day 3 with or without Vehicle or PCN (75 mg/kg) by gavage for 4 days. c, Wildtype or IRF3^{-/-} mice (n=4) were treated with 0.1% NaCl or polyI:C (150 μg) intravenous (i.v.) on Day 1 and Day 3 and Vehicle (1% DMSO, corn oil), PCN (75 mg/kg) by gavage and/or LCA (0.25 mg/kg) intraperitoneal (i.p.) for 4 days. Serum was collected and analyzed for serum alanine aminotransferase (ALT) as described in Materials and Methods. *P≦0.001 d, Representative H&E staining of livers isolated following treatment in c., arrows indicate necrotic foci.

FIG. 8: RXRα repression by polyI:C/LPS requires IRF3 but not Type 1 IFNs. a, BMMs were stimulated with LPS (10 ng/ml) or polyI:C (1 μg/ml) for 4 hrs. RNA was collected and analyzed by quantitative RT-PCR (Q-PCR). b, BMMs were stimulated with LPS (10 ng/ml) or polyI:C (1 μg/ml) for 1, 4 or 8 hrs. RNA was analyzed by Q-PCR. c, Wildtype, IRF3^{-/-} and IFNAR^{-/-} BMMs and their wildtype controls were stimulated with polyI:C (1 μg/ml) for 8 hrs. RNA was analyzed by Q-PCR. d, BMMs were stimulated with Control (DMSO), LG268 (10 nM) and GW3965 (1 μM) with or without polyI:C (1 μg/ml) for 24 hrs. Anti-RXRα and anti-USF2 Western Blot analysis was done with 75 μg of whole cell extract. e, Wildtype, IRF3^{-/-} and IFNAR^{-/-} BMMs were stimulated with Control (DMSO), LG268 (10 nM) and polyI:C (1 μg/ml) for 24 hrs. Anti-RXRα and anti-USF2 Western Blot analysis was done with 75 ug of whole cell extract. f, BMMs were stimulated with Control (DMSO) or MG132 (10 μM) with or without Control (DMSO) or 9cRA and polyI:C. Anti-RXRα and anti-USF2 Western Blot analysis was done with 75 μg of whole cell extract.

FIG. 9: PolyI:C transcriptionally represses RXRα through recruitment of transcriptional repression machinery. a, BMMs were stimulated with media or polyI:C (1 μg/ml) for 2 hrs, followed by actinomycin D (ActD) (5 μg/ml) for 0, 15, 30, 60, 120 min. BMMs were stimulated with polyI:C (1 μg/ml) for 4 hrs or 8 hrs. RNA was analyzed by Q-PCR. b, Diagram of RXRα promoter based on promoter analysis software (MatInspector). BMMs were stimulated with LPS (10 ng/ml) or polyI:C (1 μg/ml) for 4 hrs. RNA was analyzed by Q-PCR. Wildtype, IRF3^{-/-} and IFNAR^{-/-} BMMs were stimulated with polyI:C (1 μg/ml) for 4 hrs. RNA was analyzed by Q-PCR. c, pCMV-RAW 264.7 cells (MT) or pCMV-Hes1-RAW 264.7 cells (Hes1) RNA was analyzed by Q-PCR. d, RAW264.7 cells transfected with siNS or siHes1 duplex oligos were stimulated with polyI:C (1 μg/ml) for 8 hours. RNA was analyzed by Q-PCR. e, f, BMMs were stimulated with polyI:C (1 μg/ml) for 1, 3 and 6 hours. Following stimulation, chromatin immunoprecipitation (ChIP) was performed with anti-Hes1 or anti-HDAC1 antibodies on sonicated samples, washed thoroughly and analyzed by PCR/agarose gel electrophoresis. PCR products on gel are quantified by ImageJ, normalized to Input. g, BMMs were pre-treated with Trichostatin A (TSA) (50 ng/ml) overnight and then stimulated with polyI:C (1 μg/ml) for 4 and 8 hrs. RNA was analyzed by Q-PCR.

FIG. 10: PolyI:C transcriptional repression of RXRα is critical for repression of nuclear receptor target genes. a, Wildtype, IRF3^{-/-} and IFNAR^{-/-} BMMs were stimulated with Control (DMSO), or LG268 (10 nM) with or without polyI:C (1 μg/ml). RNA was analyzed by Q-PCR. b, pCMV-RAW 264.7 cells (MT) or pCMV-Hes1-RAW 264.7 cells (Hes1) were stimulated with Control (DMSO) and 9cRA with or without polyI:C (1 μg/ml) for 24 hrs. RNA was analyzed by Q-PCR. c, RAW264.7 cells transfected with siNS or siHes1 duplex oligos were stimulated with Control (DMSO) and 9cRA (10 μM) with or without polyI:C (1 μg/ml) for 24 hours. RNA was analyzed by Q-PCR. d, pBabe-RAW 264.7 cells (Raw_MT) and pBabe-RXRα-RAW264.7 cells (Raw-RXRα) were stimulated with Control (DMSO) or LG268 (10 nM) with or without polyI:C (1 μg/ml) for 24 hrs. RNA was analyzed by Q-PCR. e, pBabe-Huh7 cells (Huh7 MT) and pBabe-RXRα-Huh7 cells (Huh7-RXRα) were stimulated with Control (DMSO) and rifampicin (25 μM) with or without polyI:C (2 μg/ml, transfected). RNA was analyzed by Q-PCR. f, pBabe-Huh7 cells (Huh7 MT) and pBabe-RXRα-Huh7 cells (Huh7_RXRα) were stimulated with Control (DMSO) and ASA (20 μg/ml) with or without polyI:C (2 μg/ml, transfected). RNA was analyzed by Q-PCR. g,h, BMMs were stimulated with rifampicin (25 μM) and polyI:C (1 μg/ml) for 24 hours. Following stimulation, chromatin immunoprecipitation (ChIP) was performed with anti-RXRα antibody on sonicated samples, washed thoroughly and analyzed by PCR/agarose gel electrophoresis. PCR products on gel are quantified by ImageJ, normalized to Input.

FIG. 11: PolyI:C and Viral Infection promote acetylsalicylic acid-related hepatotoxicity through IRF3, independent of Type I IFNs. a, Representative Oil Red 0 staining of livers from wildtype (n=4) treated with 0.1% NaCl or VSV (2.5e7 pfu) intravenous (i.v.) on Day 1 with or without acetylsalicylic acid (ASA) (3.25 g/L) in drinking water for 4 days. b,c, Wildtype mice (n=4) were treated with 0.1% NaCl or VSV (2.5e7 pfu) intravenous (i.v.) on Day 1 with or without acetylsalicylic acid (ASA) (325 mg/L) in drinking water for 4 days. Serum was collected and serum ALT and blood glucose were analyzed as described in Materials and Methods. *P≦0.001 d, Representative H&E staining of livers from wildtype, IRF3^{-/-} and IFNAR^{-/-} mice treated with 0.1% NaCl or poly I:C (150 μg) intravenous (i.v.) on Day 1 and Day 3 with or without acetylsalicylic acid (ASA) (3.25 g/L) in drinking water for 4 days, arrows indicate necrotic foci. e,f, Wildtype, IRF3^{-/-} or IFNAR^{-/-} mice (n=4) were treated with 0.1% NaCl or polyI:C (150 μg) intravenous (i.v.) on Day 1 and Day 3 with or without acetylsalicylic acid (ASA) (3.25 g/L) in drinking water for 4 days. Serum was collected and serum ALT and blood glucose were analyzed as described in Materials and Methods. *P≦0.001 g, Wildtype, mice (n=4) were treated with 0.1% NaCl or VSV (2.5e7 pfu) intravenous (i.v.) on Day 1 or polyI:C (150 μg) intravenous (i.v.) on Day 1 and Day 3 with or without acetylsalicylic acid (ASA) (3.25 g/L) in drinking water for 4 days. Serum was collected and serum ammonia and total bilirubin levels were analyzed as described in Materials and Methods. *P≦0.01

FIG. 12: PolyI:C and Viral Infection inhibit acetylsalicylic acid induction of UGT1A6. a, Wildtype (n=4) were treated with 0.1% NaCl or VSV (2.5e7 pfu) intravenous (i.v.) on Day 1 with or without acetylsalicylic acid (ASA) (3.25 g/L) in drinking water for 4 days or Vehicle or PCN (75 mg/kg) by gavage for 4 days. Liver samples were isolated and RNA was analyzed by Q-PCR. b, Wildtype mice (n=4) were treated with 0.1% NaCl or polyI:C (150 μg) intravenous (i.v.) on Day 1 and Day 3 with or without acetylsalicylic acid (ASA) (3.25 g/L) in drinking water for 4 days. Liver samples were isolated and RNA was analyzed by Q-PCR. c, Representative Western blot of RXRα and USF2 from samples in b. d,e, Huh7 cells transfected with siNS or siRXRα duplex oligos were stimulated with rifampicin (25 μM) or ASA (20 μg/ml) for 24 hours. RNA was analyzed by Q-PCR.

FIG. 13: Model of IRF3-nuclear receptor crosstalk and biological consequence. Activation of IRF3 through Pattern Recognition Receptors (PRRs) results in the induction of anti-viral genes through Type I IFNs or the repression of RXRα target genes through Hes1. The repression of RXRα target genes, such as CYPs and UGTs, results in a decrease in RXR-mediated metabolism and pathogenesis of metabolic disorders such as Reye's Syndrome.

FIG. 14: Repression of hepatic nuclear receptor target genes by polyI:C/VSV. a, Wildtype (n=4) or IRF3^{-/-} (n=4) mice were treated with 0.1% NaCl or VSV (2.5e7 pfu) on Day 1 or polyI:C intravenous (i.v.) on Day 1 and Day3 with or without Vehicle (1% DMSO, corn oil) or 1,25-Dihydroxyvitamin D3 (1,25D) (7.5 mg/kg) by gavage for 4 days. Liver RNA was analyzed by Q-PCR. b, Huh7 cells were stimulated with Control (DMSO) or LXR agonist (GL, GW3965), FXR agonist (GF, GW4064), or PPARα agonist (Gα, GW409544) in the presence or absence of transfected polyI:C (1 μg/ml) for 24 hours. RNA was analyzed by Q-PCR.

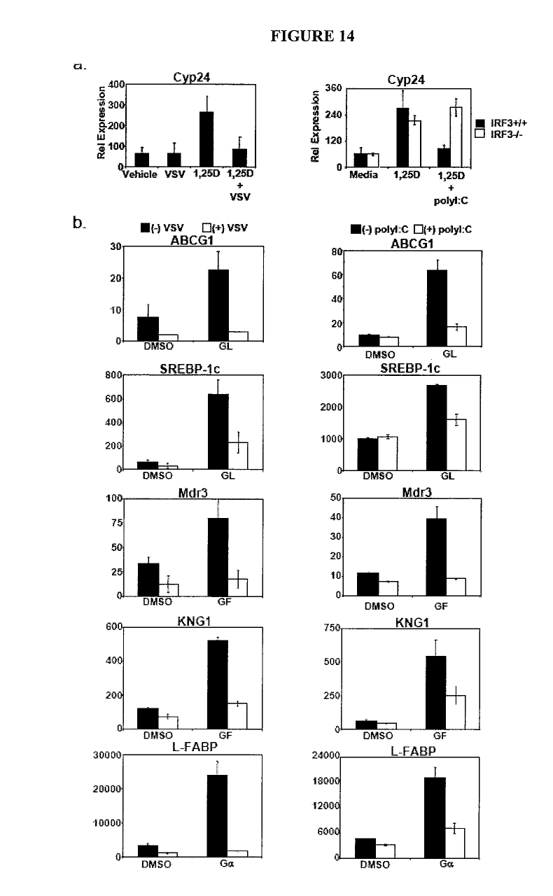

FIG. 15: PolyI:C potentiation of ASA-induced mitochondrial damage. pBabe-Huh7 cells (Huh7_MT) and pBabe-RXRα-Huh7 cells (Huh7 RXRα) were stimulated with Control (DMSO) and ASA (20 μg/ml) with or without polyI:C (1 μg/ml, transfected) for 24 hours. Cells were treated with 5 μg/ml of rhodamine 123 (Invitrogen) for 30 min, trypsinized and resuspended in PBS. Flow cytometry was done to determine rhodamine 123 uptake.

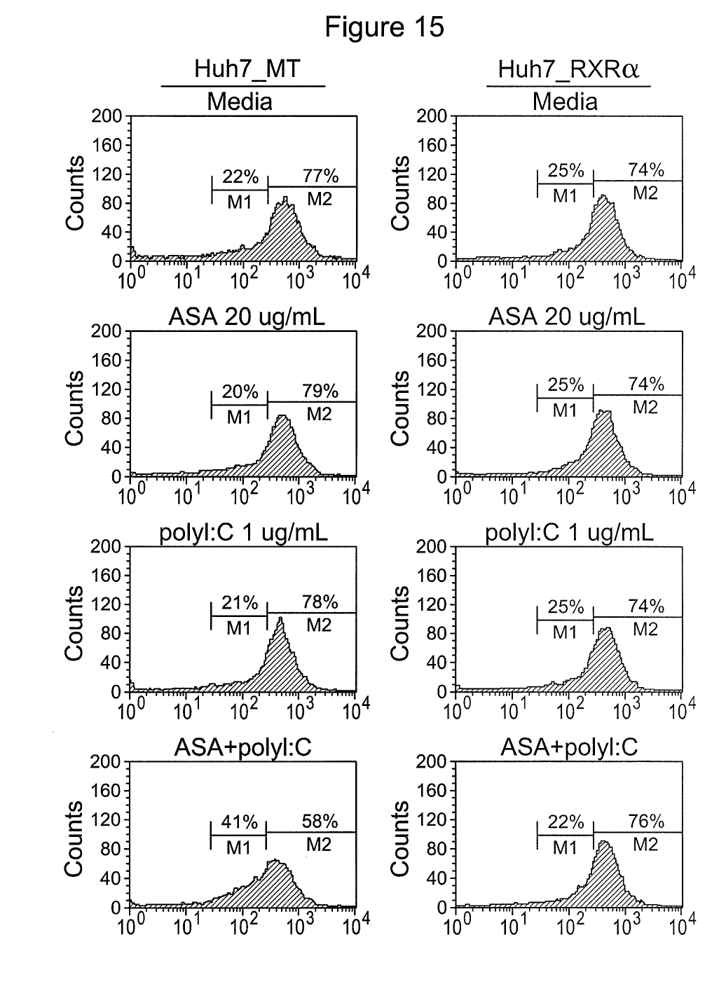

FIG. 16: RXRα protein expression in Raw-RXRα and Huh7-RXRα stable cell lines and their controls. Anti-RXRα and anti-USF2 Western Blot analysis was done with 75 μg of whole cell extract with Raw-MT and Raw-RXRα stable cell lines or Huh7-MT or Huh7-RXRα stable cell lines.

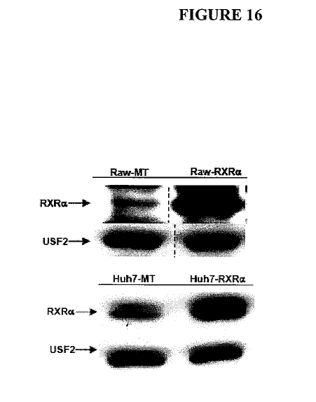

FIG. 17: PolyI:C repression of PCN induced UGT1A6 mRNA and ASA induction of PXR/RXR target genes. a, Wildtype or IRF3^{-/-} mice (n=4) were treated with 0.1% NaCl or polyI:C (150 μg) intravenous (i.v.) on Day 1 and Day 3 and Vehicle (1% DMSO, corn oil), PCN (75 mg/kg) by gavage for 4 days. Liver RNA was analyzed by Q-PCR. b, Wildtype mice (n=4) were treated with 0.1% NaCl or polyI:C (150 μg) intravenous (i.v.) on Day 1 and Day 3 with or without acetylsalicylic acid (ASA) (3.25 g/L) in drinking water for 4 days. Liver samples were isolated and RNA was analyzed by Q-PCR.

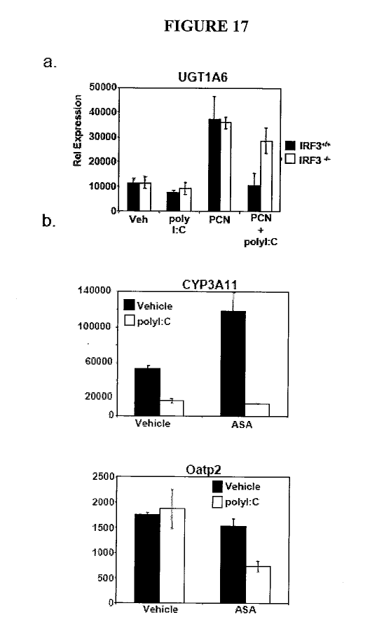

### DETAILED DESCRIPTION OF THE INVENTION

Viral infections and anti-viral responses have been linked to a number of metabolic diseases including Reye's Syndrome, which is aspirin-induced hepatotoxicity in the context of a viral infection. Here we identify an interferon regulatory factor 3 (IRF3)-dependent but type I interferon-independent pathway that strongly inhibits the expression of Retinoid X Receptor α (RXRα) and suppresses the induction of its downstream target genes including those involved in hepatic detoxification. Activation of IRF3 by viral infection in vivo greatly enhances bile acid- and aspirin-induced hepatotoxicity. This work provides a critical link between the innate immune response and host metabolism, identifying IRF3-mediated down regulation of RXRα as a molecular mechanism for pathogen-associated metabolic diseases.

In the analysis of non-Type I IFN-related roles of IRF3, we have identified a function for this factor in the repression of nuclear receptor regulated liver metabolism. We demonstrate here that activation of IRF3 during an anti-viral immune response profoundly inhibits hepatic expression of RXRα in vivo. As a consequence of this repression, the expression of multiple nuclear receptor target genes critical for xenobiotic detoxification is compromised. This pathway provides a potential molecular mechanism for the pathogenesis of Reyes' Syndrome in which acetylsalicylic acid (aspirin, ASA) treatment during a viral infection leads to hepatotoxicity. Repression of RXRα expression and downstream target genes by IRF3 represents a critical mechanism underlying metabolic diseases associated with viral infections. Accordingly, in one aspect, the invention provides means of preventing metabolic diseases associated with viral infections by administering agents which modulate the inhibition of RXRα expression by IRF3.

We have previously found that activation of IRF3 results in transcriptional repression of RXRα in a number of cell types (Chow et al., Modulation of Host Metabolism during Viral Infections through IRF3-dependent downregulation of RXRα, manuscript in submission). Repression of RXRα results in repression of nuclear receptor target genes activated by RXRα heterodimerized with a number of other nuclear receptors, including LXR, RAR, PXR and VDR.

Using acetaminophen hepatoxicity as a model system, we now have found evidence that this repression mechanism can prevent APAP hepatotoxicity. We demonstrate that polyI:C represses basal and induced levels of RXRα and CYPs involved in APAP induced hepatotoxicity. Furthermore, we demonstrate that repression of RXRα and CYPs involved IRF3. Repression of these CYPs prevents APAP from inducing serum ALT levels and cell damage in the liver. Strikingly, polyI:C was effective at increasing survival from APAP therapy at extremely high dosages. Furthermore, polyI:C was also capable of preventing APAP hepatotoxicity caused by combinatorial treatment by APAP and CYP inducers, PCN and ethanol. Thus, we have identified an extremely effective mechanism for prevention of APAP induced hepatotoxicity.

These results identify a novel mechanism that can actively protect against APAP hepatotoxicity before it occurs. Activation of IRF3 and, potentially, other transcription factors by polyI:C results in protection against APAP hepatotoxicity. We have shown that treatment with polyI:C results in lower expression of RXRα and RXRα target genes, Cyp3A11 and Cyp1A2. These CYPs are critically involved in the formation of toxic NAPQI. Lower expression of these CYPs results in an inability of APAP to increase serum ALT levels and hepatic injury. Strikingly, mice treated with polyI:C were able to survive extremely high dosages of APAP.

Besides the risk of hepatotoxicity from APAP overdose, there exists a risk of hepatotoxicity due to combinatorial ingestion of APAP and cytochrome P450 inducers. This is particularly evident in cases of APAP induced hepatotoxicity that involve alcoholics or those who have engaged in ethanol binge drinking prior or during APAP ingestion. For many of these cases, active prevention against APAP hepatotoxicity would be a much more ideal method of treatment that post APAP ingestion methods such as NAC. Our results clearly show that poly I:C is capable of protecting against APAP hepatotoxicity that results from increased sensitivity to APAP by CYP inducers such as PCN and ethanol.

The identification of an effective mechanism for protection against APAP hepatotoxicity presents the potential for the formulation of a novel APAP therapy package that would include poly I:C or an equivalent activator of IRF3 and associated transcription factors. Ideally, an equivalent or more efficient TRIF activator would potentially prove to be quite effective at regulating metabolism of APAP, allowing for greater tolerance to APAP by all individuals, including those who engage in regular usage of Cytochrome P450 inducing drugs and compounds. Use of polyI:C as a therapeutic is currently being evaluated for other uses, including ovarian and renal cancer (Adams et al. Vaccine 23, 2374-2378 (2005); Ewel et al., Cancer Res 52, 3005-3010 (1992)). PolyI:C is also being evaluated as a therapeutic for chronic fatigue syndrome and AZT-resistant HIV (Gillespie et al., In Vivo 8, 375-381 (1994); Strayer et al., Clin Infect Dis 18 Suppl 1, S88-95 (1994)). Toxicity of polyI:C has also been evaluated in a number of studies and have indicated that polyI:C can be taken at high dosages without any toxicity (Hendrix et al., Antimicrob Agents Chemother 37, 429-435 (1993)). This makes polyI:C and compounds that activate similar molecular signaling mechanisms ideal additives to APAP to prevent APAP hepatotoxicity without causing toxicity of their own. In our experiments, non-toxic levels of polyI:C were extremely effective at preventing APAP hepatotoxicity.

Thus, we have presented evidence that polyI:C activation of IRF3 and its related transcription factors is an effective mechanism for protecting against APAP induced hepatotoxicity. Furthermore, this mechanism of protection should be more effective than current treatments and would serve to prevent APAP overdose prior to identification of potential APAP overdose, a key requirement for current treatment with NAC.

Additionally, the methods are readily applicable to the prevention or treatment of toxicity associated with other compounds which are metabolized by members of the cytochrome P450 enzyme family to toxic metabolites. The methods are also readily applicable to countering the effects of the inducers of such cytochrome P450 enzyme families in increasing the conversion of a compound to a toxic metabolite.

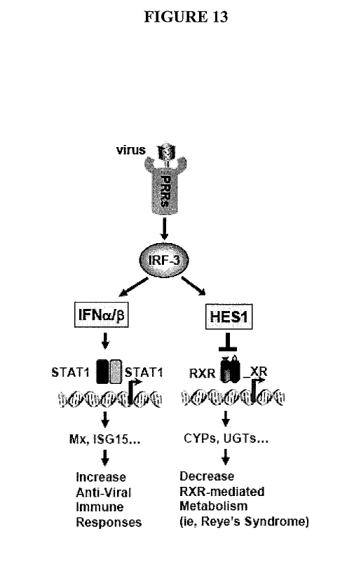

The connection between viral infections and metabolic dysfunction is an important clinical problem, yet the mechanisms linking these events had not as yet been understood. Here we provide in vivo evidence for a novel pathway linking viral infection to metabolic disease. We have shown that activation of IRF3 during the viral immune response leads to a profound suppression of RXRα mRNA and protein expression. Since RXRα serves as an obligatory heterodimeric partner for several nuclear receptors involved in metabolic control, these observations provide a molecular explanation for how viral infections can alter a range of metabolic pathways. As a consequence of RXRα suppression during viral infection, the expression of multiple downstream nuclear receptor target genes is compromised, including those required for liver detoxification of endogenous and exogenous compounds and those required for lipid metabolism. Moreover, the ability of viral infections to repress nuclear receptor function leads to hepatotoxicity in the context of endogenous toxins such as lithocholic acid and exogenous compounds such as ASA. These data provide a molecular mechanism to explain how viral infections may interfere with liver homeostasis and contribute to the pathogenesis of metabolic disease (FIG. 13).

The clinical relevance of IRF3-mediated inhibition of liver metabolism is illustrated by its potential role in the pathogenesis of hepatic metabolic disorders that involve xenobiotics (drugs and chemicals) ingested during viral infections. One such disorder, Reye's Syndrome, has yet to be explained mechanistically. It is known that ASA therapy during a viral infection in children can lead to fatty degeneration of the liver and encephalopathy (Ruben, F. L., Streiff, E. J. et al., Am J Public Health, 66:1096-1098 (1976)). Not specific to any virus in particular, Reye's Syndrome is associated with chickenpox, influenza A or B, adenoviruses, hepatitis A viruses, paramyxovirus, picornaviruses, reoviruses, herpesviruses, measles and varicella-zoster viruses (Belay at al., N Engl J Med, 340:1377-1382 (1999); Pronicka, E., Pediatr Pol, 107-110 (1999); Iwanczak et al., [2 cases of Stevens-Johnson syndrome in children], 26:1539-1542 (1973); Reye et al., Lancet, 91:749-752 (1963); Duerksen et al., Gut, 41:121-124 (1997); Orlowski et al., Cleve Clin J Med, 57:323-329 (1990); Ghosh et al., Indian Pediatr, 36:1097-1106 (1999)). Previous studies have suggested that hepatotoxicity in Reye's Syndrome results from a toxic combination of ASA metabolites and inflammatory cytokines generated in response to a viral infection (Treon, S. P. and Broitman, S. A., Med Hypotheses, 39:238-242 1992)).

It has also been shown that polyI:C can inhibit the metabolism of aspirin and this has been suggested to occur through Type I IFNs (Dolphin et al., Biochem Pharmacol, 36:2437-2442 (1987)). Our experimental model of polyI:C/VSV and ASA treatment, however, clearly demonstrates that hepatotoxicity and fatty degeneration occurs in an IRF3-dependent, Type I IFN-independent manner, consistent with those seen during Reye's Syndrome. Furthermore, it appears that this pathogenesis arises from IRF3 repression of RXRα and its hepatic target genes involved in ASA metabolism. We showed that this repression of RXRα blocks ASA and PCN induction of UGT1A6 and CYP3A11, RXR heterodimer target genes involved in ASA metabolism, and results in increased mitochondrial damage by ASA, a known contributing factor to the pathogenesis of Reye's Syndrome (Trost, L. C. and Lemasters, J. J., Toxicol Appl Pharmacol, 147:431-441 (1997); Partin et al., N Engl Med, 285:1339-1343 (1971); Martens et al, Arch Biochem Biophys, 244:773-786 (1986); Tomoda et al., Liver, 14:103-108.). Our results therefore provide compelling evidence for the involvement of IRF3-nuclear receptor crosstalk in the development of Reye's Syndrome and suggest new therapeutic strategies for the prevention of hepatotoxicity associated with viral infections.

Our results also demonstrate that viral infections can alter the clearance of endogenous toxins that accumulate during normal metabolism. LCA, a secondary bile acid produced by intestinal bacteria, is metabolized by RXR heterodimers through the induction of Cytochrome P450 family members such as CYP3A11, which catalyze the initial hydroxylation of LCA (Araya, Z. and Wikvall, K., Biochim Biophys Acta, 1438:47-54 (1999)). Mice deficient in hepatocyte PXR or RXRα exhibit functional defects in the expression of LCA metabolic genes (Xie et al., Proc Natl Acad Sci, 98:3375-3380 (2001); Staudinger et al., Proc Natl Acad Sci USA, 98:3369-3374 (2001); Wan et al., Mol Cell Biol, 20:4436-4444)). Excess amounts of LCA disturb liver homeostasis and result in cholestasis, which can be alleviated by the activation of PXR/RXR with less toxic, but more potent nuclear receptor agonists such as PCN (Xie et al., Proc Natl Acad Sci, 98:3375-3380 (2001); Staudinger et al., Proc Natl Acad Sci USA, 98:3369-3374 (2001); Wan et al., Mol Cell Biol, 20:4436-4444)). In this work, we have shown activation of IRF3 during viral infection inhibits PXR/RXR-dependent activation of CYP3A11. Consequently, viral infections render mice highly susceptible to LCA-mediated cholestasis and hepatotoxicity. Interestingly, this mechanism may be relevant to viral-induced cholestasis in humans, as EBV infections have been linked to cholestasis (Shaukat et al., Hepatol Res, ______ (2005)). The molecular pathways elucidated in our study will likely provide a useful framework for further investigation into this connection.

IRF3 is a transcription factor best known for its function in type I IFN production during the innate immune response against viral infections. Our studies have identified a new function for virally activated IRF3, repression of RXRα, that is independent of the type I IFN pathway. We have shown that activation of IRF3 induces expression of the transcriptional repressor Hes1, which binds directly to the proximal promoter of RXRα and recruits HDAC1 to repress transcription. Nevertheless, RXRα protein levels remain relatively stable in the absence of nuclear receptor activating signal. However, in combination with 26S-proteosome complex activation by nuclear agonists (ASA, PCN, LG268 and GW3965), this pathway results in a biologically significant loss of RXRα protein that would not be seen in the absence of IRF3 activation, where RXRα protein levels are replenished as new transcript is continually made. While the repression of other nuclear receptors may contribute to our observed phenomenoms, mutation of RXRα in hepatocytes results in similar in vivo defects in PXR/RXR target gene induction and increased LCA sensitivity as seen in our studies with polyI:C and VSV, providing further evidence that IRF3-mediated down regulation of RXRα could contribute significantly to the pathogenesis of hepatic metabolic diseases (Wan et al., Mol Cell Biol, 20:4436-4444 (2000)). Previous work has shown that nuclear receptor activation can inhibit IRF3 target genes (Ogawa et al., Cell, 122:707-721 (2005)). It is possible that the down regulation of RXRα may relieve this inhibitory effect and allow for optimal induction of IRF3 target genes involved in anti-viral response. However, it is not clear whether this RXRα down regulation will be overall beneficial or harmful to the host during a microbial infection.

The central role of RXRα in nuclear receptor signaling indicates that IRF3-nuclear receptor crosstalk may have implications for a variety of pathways and metabolic functions. The particular importance of the RXRα isoform is clear in that RXRα-deficient mice are embryonic lethal (Sucov et al., Genes Dev, 8:1007-1018 (1994); Kastner et al., Cell, 78:987-1003 (1994)). Furthermore, a number of tissue specific RXRα-deficient mice have been described that point to diverse functions for this receptor (Imai et al., Proc Natl Acad Sci USA, 98:224-228 (2001); Li et al., Nature, 407:633-636 *(2001); Wan et al., Mol Cell Biol, 20:4436-4444.40 (2000)). Loss of RXRα has been demonstrated in our work and others to inhibit some RXR heterodimer target genes, but not all, suggesting that other factors may play overlapping roles in determining activation and maintenance of certain nuclear receptor target genes (Castrillo et al., Mol Cell, 12:805-816 (2003); Wan et al., Mol Cell Biol 20:4436-4444 (2000)). However, it is clear from our work and these genetic studies of RXRα, loss of RXRα would affect a number of nuclear receptor pathways. Thus, in addition to contributing to the pathogenesis of Reye's Syndrome, IRF3 repression of RXRα may contribute to other diseases associated with viral infections. One such disease is atherosclerosis, where IRF3 activation contributes to negative regulation of LXR-related genes and cholesterol efflux (Castrillo et al., Mol Cell, 12:805-816 (2003)). These results indicate that IRF3-dependent down regulation of RXRα influences disorders such as Gianotti-Crosti Syndrome in the skin (Ratziu et al., Aliment Pharmacol Ther, 22 Suppl 2:56-60 (2005); Yoshida et al., J Pediatr, 145:843-844 (2004)) and viral-linked diabetes (Ratziu et al., Aliment Pharmacol Ther, 22 Suppl 2:56-60 (2005)). IRF3-nuclear receptor crosstalk provides a new understanding of the link between microbial infection and metabolic dysfunction and suggests novel targets for therapeutic intervention in these syndromes.

### DEFINITIONS

Unless otherwise stated, the following terms used in the specification and claims have the meanings given below.

It is noted here that as used in this specification and the appended claims, the singular forms “a,” “an,” and “the” include plural reference unless the context clearly dictates otherwise.

Modulators are agents which can increase or decrease a referenced activity. Modulators include inhibitors and activators. Activators generally act opposite to inhibitors (e.g., increase, stimulate, augment, enhance, accelerate) a referenced activity or entity. A modulator of an identified protein can be an activator or inhibitor of the protein, additionally, the modulator can be an agent which modulates the expression of the protein, or the levels of the protein in a tissue (e.g., liver, lung, kidney, intestinal lining).

An IRF3 polypeptide according to the invention is a mammalian IRF3 protein, preferably, wild-type, and more preferably, human (see, SEQ ID NO:1). The protein can be activated or unactivated by phosphorylation. When activated, the IRF3 protein acts to suppress or inhibit expression or levels of RXRα. With regard to amino acid sequence, an IRF3 polypeptide according to the invention 1) comprises, consists of, or consists essentially of an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of over a region of at least about 15, 20, 25, 50, 75, 100, 125, 150 or more amino acids, to a polypeptide of Table 1 (SEQ ID NO:1); retains a specific biological binding activity of IRF3 or can specifically bind to an antibody, e.g., polyclonal antibody, raised against an epitope of IRF3. In some embodiments, the IRF3 polypeptide is a fragment of IRF3 is an N-terminal, C-terminal, or midportion of IRF3 comprising 95, 95, or 99% of the full sequence.

The terms “identical” or percent “identity,” in the context of two or more nucleic acids or polypeptide sequences, including IRF3 polypeptides, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to the full length of the reference sequence, usually about 25 to 100, or 50 to about 150, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA, 89:10915 (1989)) alignments (B) of 50, expectation (E) of 10, M=5, N=−4, and a comparison of both strands.

An IRF3 polypeptide according to the invention may be a conservatively modified variant of a polypeptide of SEQ ID NO:1. Accordingly, in some embodiments of the above, the IRF3 polypeptide consists of the sequence of IRF3 of SEQ ID NO:1 or a fragment thereof. The fragment may be from 15 to 25, 15 to 40, 25 to 50, 50 to 100 amino acids long, or longer. The fragment may correspond to that of IRF3. In some other embodiments still, the IRF3 polypeptide sequence can be that of a mammal including, but not limited to, primate, e.g., human; rodent, e.g., rat, mouse, hamster; cow, pig, horse, sheep. The proteins of the invention include both naturally occurring or recombinant molecules. In some embodiments, the amino acids of the IRF3 polypeptide are all naturally occurring amino acids as set forth below. In other embodiments, one or more amino acids may be substituted by an artificial chemical mimetic of a corresponding naturally occurring amino acids.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Methods for obtaining (e.g., producing. isolating, purifying, synthesizing, and recombinantly manufacturing) polypeptides are well known to one of ordinary skill in the art.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As to “conservatively modified variants” of amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

An “anti-IRF3 antibody” or “IRF3 antibody” according to the invention is an antibody which can bind to the IRF3 polypeptide of SEQ ID NO:1. The antibodies according to the invention can act to inhibit the biological activity of IRF3 in influencing cytochrome P450 enzyme expression or levels. The IRF3 modulatory antibodies for use according to the invention include, but are not limited to, recombinant antibodies, polyclonal antibodies, monoclonal antibodies, chimeric antibodies, human monoclonal antibodies, humanized or primatized monoclonal antibodies, and antibody fragments. In some embodiments, the antibodies bind to a wild-type mammalian IRF3.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al, Nature 348:552-554 (1990))

For preparation of antibodies, e.g., recombinant, monoclonal, or polyclonal antibodies, many techniques known in the art can be used (see, e.g., Kohler & Milstein, Nature 256:495-497 (1975); Kozbor et al, Immunology Today, 4:72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies. Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology, (3^{rd} ed. 1997)). Techniques for the production of single chain antibodies or recombinant antibodies (U.S. Pat. No. 4,946,778, U.S. Pat. No. 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016; Marks et al., Bio/Technology, 10:779-783 (1992); Lonberg, et al., Nature, 368:856-859 (1994); Morrison, Nature 368:812-13 (1994); Fishwild et al., Nature Biotechnology, 14:845-51 (1996); Neuberger, Nature Biotechnology, 14:826 (1996); and Lonberg & Huszar, Intern. Rev. Immunol., 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology, 10:779-783 (1992)). Antibodies can also be made bispecific, i.e., able to recognize two different antigens (see, e.g., WO 93/08829, Traunecker et al., EMBO J., 10:3655-3659 (1991); and Suresh et al., Methods in Enzymology, 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Jones et al., Nature, 321:522-525 (1986); Riechmann et al., Nature, 332:323-327 (1988); Verhoeyen et al., Science, 239:1534-1536 (1988) and Presta, Curr. Op. Struct. Biol., 2:593-596 (1992)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only those polyclonal antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998), for a description of immunoassay formats and conditions that can be used to determine specific immunoreactivity).

For example, rabbit polyclonal antibodies are known in the art (see, Wang et al., Blood, 97:3890-3895 (2001)). Such antibodies may be obtained using glutathione-S-transferase-IRF3 fusion proteins. Rabbit antibodies can be generated against the first extracellular region of the gene (from amino acid 16 to 64) constructed as a glutathione-S-transferase (GST)-IRF3 fusion protein. The IRF3 peptide can be cloned by PCR using the following primers corresponding to an IRF3 nucleic acid sequence (see, for instance, SEQ ID NO:2). The PCR product can be directionally cloned into the BamHI and EcoRI sites of the pGEX-4T-1 vector that contains GST gene (Pharmacia). The IRF3 fragment can be cloned in frame with the GST to create a fusion protein. The insert can be confirmed by sequencing. The GST fusion protein can be produced as previously described (see, Smith, D. B. et al., Gene, 67:31-40 (1988)). Bacteria in log phase (OD₆₀₀ 0.6 to 0.9) can be induced for 2.5 to 3 hours at 37° C. with 1 mM isopropyl-1-thio-β-D-galactopyranoside. Bacteria are lysed, and the soluble fraction loaded onto a glutathione-Sepharose column (Pierce, Rockford, Ill.). The columns are washed with 10 bed volumes of phosphate-buffered saline (PBS)/EDTA. The fusion protein elutes from the column using 20 mM reduced glutathione (Sigma, St Louis, Mo.) in 50 mM Tris-Cl, pH 8.0. For antibody preparation, rabbits are immunized twice with the GST-IRF3 fusion protein, and serum is collected, starting two weeks after the last immunization (Research Genetics, Huntsville, Ala.).

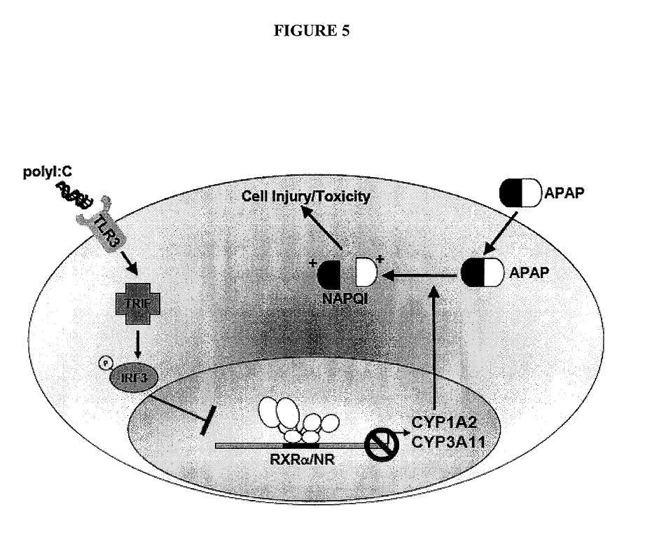

IRF3 modulators which increase or decrease the levels or activity or expression of IRF3 can be useful in different aspects of the invention. For xenobiotics or other compounds which are detoxified by a Cytochrome P450 enzyme, infection and the resulting increased IRF3 levels, expression, or activity can lead to an increased toxicity of the compound by reducing the levels, expression or activity of the Cytochrome P450 enzyme responsible for its removal/detoxification. For these compounds in the case of infection, administration of an IRF3 inhibitor or antagonist to a subject can be protective. Conversely, for those xenobiotics whose toxicity is increased as a result of metabolism by a cytochrome P450 enzyme, administration of an IRF3 agonist or activator can be useful in reducing its toxicity, especially, in the presence of an inducer for the enzyme. Preferred IRF3 activators for use in the invention are poly I:C, poly C:G, double-stranded RNA, imidazoquinoline or R848, and Toll receptor agonists. In some embodiments, the IRF3 modulator is a TRIF or TLR3 modulator in an IRF3 activation pathway of FIG. 5. Upon viral infection or stimulation with toll-like receptor agonists such as polyI:C or LPS, IRF3 is phosphorylated by serine/threonine kinase, TANK binding kinase 1 (TBK1) or Inducible IκB kinase (IKKi) (Perry et al., J Exp Med, 199:1651-1658 (2004)). Accordingly, modulators of TBK1 or IKKi may also be used to modulate IRF3. In addition to being activated by TLR-TRIF-dependent pathways (Yamamoto et al., Science, 301:640-643 (2003)), intracellular receptors such as RIG-I are capable of activating IRF3 upon recognition of polyI:C and RNA viruses (Li et al., J Biol Chem, 280:16739-16747; Yoneyama et al., Nat Immunol, 5:730-737 (2004)). Accordingly, modulators of RIG-I are also suitable modulators of IRF3. Following activation, IRF3 promotes transcription of Type I IFN genes together with other transcription factors such as NF-κB and AP-1 (Perry et al., J Exp Med, 199:1651-1658 (2004); Fitzgerald et al., J Exp Med, 198:1043-1055.(2003); Jiang et al., Proc Natl Acad Sci USA, 101:3533-3538 (2004)).

Additional toll receptor ligands for use as IRF3 modulators include those listed in the following Table A which sets forth exemplary TLR receptors and modulators of IRF3:

TABLE A
IRF3 Modulators

TLR1: Borrelia burgdorferi, neisseria, lipoproteins (mycobacteria);
triacyl lipopeptides (synthetic analogue).
TLR2: Trypanosomes, mycoplasma, borrelia, listeria, klebsiella,
herpes simplex virus, zymosan (yeast), lipoteichoic acid and
peptidoglycan (Gram+), lipoproteins (mycobacteria), atypical
lipopolysaccharide (Gram−), glycolipids, lipoarabinomannan,
HSP 60 and HSP 70 (endogenous ligand); di- and triacyl lipopeptides
(synthetic analogue). Porins, defensins, Pam3Cys.
TLR3: Viral double-stranded RNA; Poly I:C (synthetic analogue),
endogenous mRNA
TLR4: Plant product taxol, mycobacteria, respiratory syncytial
virus, fibrinogen peptides, fibronecti,n bacterial lipopoly-
saccharides (Gram−), HSP60 (endogenous ligand), HSP70, HSP 90;
lipopolysaccharide/lipid A mimetics (synthetic analogue); synthetic
lipid A, E5564 (fully synthetic small molecule), MMTV, Heparin
sulfate, Hyaluronic acid, defensins, Pseudomonas exoenxyme S.
TLR5: Bacterial flagellins; discontinuous 13-amino-acid peptide
(synthetic analogue)
TLR6: Zymosan (fungi), lipopeptides (mycoplasma), lipotechoic acid;
diacyl lipopeptides (synthetic analogue).
TLR7: Single-stranded RNA, R-837 and R848; imidazole quinolines, i.e.
Imiquimod, Resiquimod (fully synthetic small molecule); guanosine
nucleotides, i.e. loxoribine (fully synthetic small molecule).
TLR8: Single-stranded RNA, R848; imidazole quinolines, i.e. Imiquimod
(fully synthetic small molecule)
TLR9: Bacterial DNA, viral DNA, other DNA with low content of non-
methylated CpG sequences; CpG oligonucleotides (synthetic analogue).
TLR10
TLR11: Bacterial components from uropathogenic bacteria
TLR12:
TLR13

Accordingly, methods of prevention and alleviation of hetatotoxicity induced by acetaminophen (APAP) and other toxic compounds include administration of agents which modulate portions or members of the TLR3 to IRF3 pathway of FIG. 5. Such agents include, but are not limited to small molecules, natural or synthetic ligands, antibodies and cDNAs for targets that can enhance IRF3-mediated repression of RXRα. Such potential targets include but are not limited to Toll-like receptors (TLR1, TLR2, TLR3, TLR4, TLR5, TLR6, TLR7, TLR8, TLR9, TLR10, TLR11, TLR12, TLR13), RIG-I like receptors (RIG-I and Mda-5), CARDIF, MyD88 family members (MyD88, TRIF, SARM), TRAF family members (TRAF6 and TRAF3), IKK family members (TBK1, IKKi, IKKα, IKKβ), IRF3 family members (IRF3 and IRF7), and Hes1.

Methods of prevention and alleviation of hetatotoxicity associated with infections include administration of modulators of portions or members of the TLR3 to IRF3 pathway of FIG. 5. Such modulators include, but are not limited to, small molecules, natural or synthetic antagonists, antibodies, cDNA fragments and SiRNA for targets that can prevent IRF3-mediated repression of RXRα. Such potential targets include but are not limited to Toll-like receptors (TLR1, TLR2, TLR3, TLR4, TLR5, TLR6, TLR7, TLR8, TLR9, TLR10, TLR11, TLR12, TLR13), RIG-I like receptors (RIG-I and Mda-5), CARDIF, MyD88 family members (MyD88, TRIF, SARM), TRAF family members (TRAF6 and TRAF3), IKK family members (TBK1, IKKi, IKKα, IKKβ), IRF3 family members (IRF3 and IRF7), and Hes1.

Accordingly, administration of an IRF3 activator or agonist can reduce the expression, activity or tissue levels of members of the Cytochrome P450 enzyme family. In some embodiments, the IRF3 activator reduces the expression, activity, or levels of a member of the enzyme family involved in the metabolism of a toxic compound of interest. In some embodiments, the cytochrome P450 enzyme is one or both of Cytochrome P450 3A11 or Cytochrome P450 1A2. In still other embodiments, the cytochrome P450 enzyme comprises a Cytochrome P450 isoform selected from Cytochrome P450 1A2, Cytochrome P450 2B6, Cytochrome P450 2C19, Cytochrome P450 2C9, Cytochrome P450 2D6, Cytochrome P450 2E1, and Cytochrome P450 3A 4, 5, or 7. In exemplary embodiments, the CYP is a liver CYP. However, as CYP is found in other tissues and actively metabolizes xenobiotic and other compounds in those tissues, in some embodiments, epithelial or other tissue, for instance, of the lung, kidney, or intestine.

In some embodiments, the IRF3 modulatory agent is poly C:G or a lipopolysaccharide (LPS) or a gram negative bacterial LPS. LPS are generally distinguished by a lipid A moiety, in which primary and secondary acyl chains are linked to a disaccharide-phosphate backbone, a ketodeoxyoctulosonic acid moiety, and a polysaccharide moiety of highly variable structure.

Poly I:C or polyinosinic: polycytidylic acid is a very high molecular weight (e.g., weights in excess of one million Daltons) co-polymer of 5′-inosinic acid, homopolymer complexed with a 5′-cytidylic acid homopolymer (1:1). This synthetic double-stranded RNA that has often been used experimentally to model viral infections in vivo.

Acetaminophen is a well-known pain reliever and fever suppressant. The maximum daily dose of acetaminophen is about 4 g in adults and about 90 mg/kg in children. A single acute toxic ingestion is about 150 mg/kg or approximately 7 g in adults. The at-risk dose may be lower in susceptible patient populations, such as persons with alcohol abuse or malnutrition. In acute overdose or when the maximum daily dose is exceeded over a prolonged period, the normal conjugative pathways of metabolism become saturated. Excess acetaminophen is then oxidatively metabolized in the liver by the mixed function oxidase P450 system to a toxic metabolite, N-acetyl-p-benzoquinone-imine (NAPQI). NAPQI is rapidly conjugated with glutathione. In cases of excessive NAPQI formation, as in overdosage or increased mixed function oxidase metabolism or reduced glutathione stores, NAPQI can covalently bind to vital proteins and other constituents of hepatocytes resulting in severe liver damage, including hepatocellular death and centrilobular liver necrosis.

Compounds whose metabolism is to be altered by an IRF3 modulator can be a drug, a naturally occurring compound, a synthetic compound, or a xenobiotic compound not normally found in nature. Compounds which are toxic and metabolized by Cytochrome P450 can be are readily known to one of ordinary skill in the art. RTECS references many such compounds. RTECS (NIOSH 1980 or later editions, including 1995), also known as Registry of Toxic Effects of Chemical Substances, is a database of toxicity information compiled from the published scientific literature. Prior to 2001, RTECS was maintained by US National Institute for Occupational Safety and Health (NIOSH). Now it is maintained by Elsevier MDL.

The term “test compound” or “drug candidate” or “IRF3 modulator” or grammatical equivalents as used herein describes any molecule, either naturally occurring or synthetic, e.g., protein (e.g., IRF3 antibody or fragment thereof), oligopeptide (e.g., from about 5 to about 25 amino acids in length, preferably from about 10 to 20 or 12 to 18 amino acids in length, preferably 12, 15, or 18 amino acids in length), small organic molecule, polysaccharide, lipid, fatty acid, polynucleotide, RNAi, siRNA oligonucleotide, etc. The test compound can be in the form of a library of test compounds, such as a combinatorial or randomized library that provides a sufficient range of diversity. Test compounds are optionally linked to a fusion partner, e.g., targeting compounds, rescue compounds, dimerization compounds, stabilizing compounds, addressable compounds, and other functional moieties. Conventionally, new chemical entities with useful IRF3 modulatory properties are generated by identifying a test compound (called a “lead compound”) with some desirable property or activity, e.g., inhibiting activity, creating variants of the lead compound, and evaluating the property and activity of those variant compounds. Often, high throughput screening (HTS) methods are employed for such an analysis.

IRF3 modulators are preferably small organic molecules. A “small organic molecule” refers to an organic molecule, either naturally occurring or synthetic, that has a molecular weight of more than about 50 Daltons and less than about 2500 Daltons, preferably less than about 2000 Daltons, preferably between about 100 to about 1000 Daltons, more preferably between about 200 to about 500 Daltons.

An “agonist” refers to an agent that binds to a polypeptide or polynucleotide and stimulates, increases, activates, facilitates, enhances activation, sensitizes or up regulates the activity or expression of the polypeptide or polynucleotide of the invention.

An “antagonist” refers to an agent that inhibits expression of a polypeptide or polynucleotide of the invention or binds to, partially or totally blocks stimulation, decreases, prevents, delays activation, inactivates, desensitizes, or down regulates the activity of a polypeptide or polynucleotide of the invention.

“Inhibitors,” “activators,” and “modulators” of expression or of activity are used to refer to inhibitory, activating, or modulating molecules, respectively, identified using in vitro and in vivo assays for expression or activity, e.g., ligands, agonists, antagonists, and their homologs and mimetics. As mentioned above, the term “modulator” includes inhibitors and activators. Inhibitors are agents that, e.g., inhibit expression of a polypeptide or polynucleotide or bind to, partially or totally block stimulation or enzymatic activity, decrease, prevent, delay activation, inactivate, desensitize, or down regulate the activity of a polypeptide or polynucleotide, e.g., antagonists. Activators are agents that, e.g., induce or activate the expression of a polypeptide or polynucleotide or bind to, stimulate, increase, open, activate, facilitate, enhance activation or enzymatic activity, sensitize or up regulate the activity of a polypeptide or polynucleotide, e.g., agonists. Modulators include naturally occurring and synthetic ligands, antagonists, agonists, small chemical molecules and the like. Assays to identify inhibitors and activators include, e.g., applying putative modulator compounds to cells, in the presence or absence of a polypeptide or polynucleotide of the invention and then determining the functional effects on a polypeptide or polynucleotide of the invention activity. Samples or assays comprising a polypeptide or polynucleotide that are treated with a potential activator, inhibitor, or modulator are compared to control samples without the inhibitor, activator, or modulator to examine the extent of effect. Control samples (untreated with modulators) are assigned a relative activity value of 100%. Inhibition is achieved when the activity value of a polypeptide or polynucleotide of the invention relative to the control is about 80%, optionally 50% or 25-1%. Activation is achieved when the activity value of a polypeptide or polynucleotide of the invention relative to the control is 110%, optionally 150%, optionally 200-500%, or 1000-3000% or higher.

### Methods of Treatment

The terms “treating” or “treatment” of includes:

(1) preventing toxicity, i.e., causing the clinical symptoms of the toxicity not to develop in a mammal that may be exposed to the toxic compound or drug but does not yet experience or display symptoms of the disease,

(2) inhibiting the toxicity, i.e., arresting or reducing the development of the toxicity or its clinical symptoms, or eliminating the toxicity.

### Methods of Administration and Formulation

The IRF3 modulators (i.e., active agents) and their pharmaceutical compositions according to the invention may be administered by any route of administration (e.g., intravenous, topical, intraperitoneal, parenteral, oral, rectal) to treat a subject. They may be administered as a bolus or by continuous infusion over a period of time, by intramuscular, intraperitoneal, intravenous, subcutaneous, intra-articular, oral, topical, or inhalation routes. Intravenous or subcutaneous administration is preferred. The administration may be systemic. They may be administered to a subject who has been exposed, will be potentially exposed, or more particularly overexposed to a toxic compound; to a subject who has been dosed, overdosed, or suspected of being overdosed with a drug. In some embodiments, the methods include the step of first determining whether the subject was likely to be exposed or overdosed. The compound or drug is one whose toxifying metabolism is reduced by administration of the agent or the composition.

The active agents, including but not limited to Toll-receptor activators or agonists, and IRF3 activators or agonists (e.g., poly I:C, and LPS) for use according to the invention can be administered to a subject in accord with known methods, such as intravenous administration, e.g., as a bolus or by continuous infusion over a period of time, by intramuscular, intraperitoneal, intracerobrospinal, subcutaneous, intra-articular, intrasynovial, intrathecal, oral, topical, or inhalation routes. Intravenous or subcutaneous administration of biopolymers is preferred. The administration may be local or systemic.

The compositions for administration will commonly comprise the active agent as described herein dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the patient's needs.

Thus, a typical pharmaceutical composition for intravenous administration will vary according to the active agent. Actual methods for preparing parenterally administrable compositions will be known or apparent to those skilled in the art and are described in more detail in such publications as Remington: The Science and Practice of Pharmacy, 20th ed., Lippincott, Williams, and Wilkins, (2000).

The pharmaceutical compositions can be administered in a variety of unit dosage forms depending upon the method of administration. For example, unit dosage forms suitable for oral administration include, but are not limited to, powder, tablets, pills, capsules and lozenges. It is recognized that antibodies when administered orally, should be protected from digestion. This is typically accomplished either by complexing the molecules with a composition to render them resistant to acidic and enzymatic hydrolysis, or by packaging the molecules in an appropriately resistant carrier, such as a liposome or a protection barrier. Means of protecting agents from digestion are well known in the art.

Pharmaceutical formulations, particularly, of the nucleic acids, LPS and activators or agonists for use with the present invention can be prepared by mixing the agent having the desired degree of purity with optional pharmaceutically acceptable carriers, excipients or stabilizers. Such formulations can be lyophilized formulations or aqueous solutions. Acceptable carriers, excipients, or stabilizers are nontoxic to recipients at the dosages and concentrations used. Acceptable carriers, excipients or stabilizers can be acetate, phosphate, citrate, and other organic acids; antioxidants (e.g., ascorbic acid) preservatives low molecular weight polypeptides; proteins, such as serum albumin or gelatin, or hydrophilic polymers such as polyvinylpyllolidone; and amino acids, monosaccharides, disaccharides, and other carbohydrates including glucose, mannose, or dextrins; chelating agents; and ionic and non-ionic surfactants (e.g., polysorbate); salt-forming counter-ions such as sodium; metal complexes (e.g., Zn-protein complexes); and/or non-ionic surfactants.

The formulation may also provide additional active compounds, including, therapeutic agents whose metabolism is to be modulated by the agent. The active ingredients may also prepared as sustained-release preparations (e.g., semi-permeable matrices of solid hydrophobic polymers (e.g., polyesters, hydrogels (for example, poly(2-hydroxyethylmethacrylate), or poly(vinylalcohol)), polylactides. The antibodies and immunoconjugates may also be entrapped in microcapsules prepared, for example, by coacervation techniques or by interfacial polymerization, for example, hydroxymethylcellulose or gelatin microcapsules and poly-(methylmethacylate) microcapsules, respectively, in colloidal drug delivery systems (for example, liposomes, albumin microspheres, microemulsions, nano-particles and nanocapsules) or in macroemulsions.

The compositions can be administered for therapeutic or prophylactic treatments. In therapeutic applications, compositions are administered to a subject in need of treatment (e.g., suspected of exposure or dosing, or actually exposed to or administered a xenobiotic whose metabolism is to be modulated) in a “therapeutically effective dose.” Amounts effective for this use will depend upon the compound, the cytochrome P450 enzyme involved in the metabolic pathway to be modulated. Single or multiple administrations of the compositions may be administered depending on the dosage and frequency as required and tolerated by the subject. A “patient” or “subject” for the purposes of the present invention includes both humans and other animals, particularly mammals. Thus the methods are applicable to both human therapy and veterinary applications. In the preferred embodiment the patient is a mammal, preferably a primate, and in the most preferred embodiment the patient is human. Other known therapies can be used in combination with the methods of the invention. For example, the compositions for use according to the invention may also be used with N-acetylcysteine or other antidotes to the toxic agent or its metabolite.

The combined administrations contemplates coadministration, using separate formulations or a single pharmaceutical formulation, and consecutive administration in either order, wherein preferably there is a time period while both (or all) active agents simultaneously exert their biological activities.

Formulations suitable for oral administration can consist of (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

The compositions of the present invention may be sterilized by conventional, well-known sterilization techniques or may be produced under sterile conditions. Aqueous solutions can be packaged for use or filtered under aseptic conditions and lyophilized, the lyophilized preparation being combined with a sterile aqueous solution prior to administration. The compositions can contain pharmaceutically or physiologically acceptable auxiliary substances as required to approximate physiological conditions, such as pH adjusting and buffering. agents, tonicity adjusting agents, wetting agents, and the like, e.g., sodium acetate, sodium lactate, sodium chloride, potassium chloride, calcium chloride, sorbitan monolaurate, and triethanolamine oleate.

The compound of choice, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the compound of choice with a base, including, for example, liquid triglycerides, polyethylene glycols, and paraffin hydrocarbons.

Formulations suitable for parenteral administration, such as, for example, by intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration, oral administration, and intravenous administration are the preferred methods of administration. The formulations of compounds can be presented in unit-dose or multi-dose sealed containers, such as ampules and vials.

Injection solutions and suspensions can be prepared from sterile powders, granules, and tablets of the kind previously described.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents.

Preferred pharmaceutical preparations deliver one or more agents.

In therapeutic use, the active agent utilized in the pharmaceutical method of the invention are administered at the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound being employed. For example, dosages can be empirically determined considering the compound and or cytochrome P450 enzyme to be modulated. The dose administered to a patient, in the context of the present invention should be sufficient to effect a beneficial therapeutic or protective response in the patient over time. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired.

The pharmaceutical preparations for use according to the invention are typically delivered to a mammal, including humans and non-human mammals. Non-human mammals treated using the present methods include domesticated animals (i.e., canine, feline, murine, rodentia, and lagomorpha) and agricultural animals (bovine, equine, ovine, porcine).

### Assays for Modulators of IRF3, RXR Levels, and Cytochrome P450 Levels

Modulation of IRF3 can be assessed using a variety of in vitro and in vivo assays, including cell-based models. Such assays can be used to test for inhibitors and activators of a IRF3 protein, and, consequently, inhibitors and activators of RXRα expression and expression of members of the cytochrome P450 enzyme system. Such modulators have the potential to modulate the toxicity of xenobiotic in infected or uninfected mammals. Modulators of IRF3 can be studied using methods set forth in the Examples as well as by IRF3 binding assays. IRF3 protein used can be either recombinant or naturally occurring. The effect on Cytochrome P450 enzyme levels can be measured using enzyme assays for the particular enzyme, or by detecting the enzymes themselves, or by measuring their mRNA levels.

Measurement of IRF3 modulation by a candidate modulator can be performed using a variety of assays, in vitro, in vivo, and ex vivo, as described herein. A suitable physical, chemical or phenotypic change that affects activity, e.g., enzymatic activity such as kinase activity, cell proliferation, or ligand binding can be used to assess the influence of a test compound on the IFR3. When the functional effects are determined using intact cells or animals, one can also measure a variety of effects, such as, ligand binding, kinase activity, transcriptional changes to both known and uncharacterized genetic markers (e.g., northern blots), changes in cell metabolism, changes related to cellular proliferation, cell surface marker expression, histocytochemistry, apoptosis, cell death, functional loss, DNA synthesis, marker and dye dilution assays (e.g., GFP and cell tracker assays).

### In Vitro Assays

Assays to identify compounds with IRF3 modulating activity can be performed in vitro. Such assays can use a full length IRF3 protein or a variant thereof, or a mutant thereof, or a fragment of IRF33. Purified recombinant or naturally occurring IRF3 protein can be used in the in vitro methods of the invention. As described below, the binding assay can be either solid state or soluble. Preferably, the protein or membrane is bound to a solid support, either covalently or non-covalently. Often, the in vitro assays of the invention are substrate or ligand binding or affinity assays, either non-competitive or competitive. Other in vitro assays include measuring changes in spectroscopic (e.g., fluorescence, absorbance, refractive index), hydrodynamic (e.g., shape), chromatographic, or solubility properties for the protein. Other in vitro assays include enzymatic activity assays, such as phosphorylation or autophosphorylation assays).

In one embodiment, a high throughput binding assay is performed in which the IRF3 protein or a fragment thereof is contacted with a potential modulator and incubated for a suitable amount of time. In one embodiment, the potential modulator is bound to a solid support, and the IRF3 is added. In another embodiment, the IRF3 is bound to a solid support. A wide variety of modulators can be used, as described below, including small organic molecules, peptides, antibodies, and IRF3 ligand analogs. A wide variety of assays can be used to identify IRF3 modulator binding, including labeled protein-protein binding assays, electrophoretic mobility shifts, immunoassays, enzymatic assays such as kinase assays, and the like. In some cases, the binding of the candidate modulator is determined through the use of competitive binding assays, where interference with binding of a known ligand or substrate is measured in the presence of a potential modulator.

In one embodiment, microtiter plates are first coated with either an IRF3 protein or an IRF3 protein receptor, and then exposed to one or more test compounds potentially capable of inhibiting the binding of IRF3 to its receptor. A labeled (i.e., fluorescent, enzymatic, radioactive isotope) binding partner of the coated protein, either a IRF3 protein receptor or a IRF3 protein, is then exposed to the coated protein and test compounds. Unbound protein is washed away as necessary in between exposures to a IRF3 protein or a test compound. The presence or absence of a detectable signal (i.e., fluorescence, colorimetric, radioactivity) indicates that the test compound did not inhibit the binding interaction between IRF3 and its receptor. The presence or absence of detectable signal is compared to a control sample that was not exposed to a test compound, which exhibits uninhibited signal. In some embodiments the binding partner is unlabeled, but exposed to a labeled antibody that specifically binds the binding partner.

### Cell-Based In Vivo Assays

In another embodiment, IRF3 is expressed in a cell type of interest (e.g., hepatocytes), and functional, e.g., physical and chemical or phenotypic, changes are assayed to identify IRF3 modulators of RXRα activity or cytochrome P450 activity. Cells expressing IRF3 proteins can also be used in binding assays and enzymatic assays. Any suitable functional effect can be measured, as described herein. For example, cellular morphology (e.g., cell volume, nuclear volume, cell perimeter, and nuclear perimeter in response to a xenobiotic), ligand binding, kinase activity, apoptosis, cell surface marker expression, cellular proliferation, GFP positivity and dye dilution assays (e.g., cell tracker assays with dyes that bind to cell membranes), DNA synthesis assays (e.g., ³H-thymidine and fluorescent DNA-binding dyes such as BrdU or Hoechst dye with FACS analysis), are all suitable assays to identify potential modulators using a cell based system, especially in the presence of a xenobiotic whose toxicity to the cell is being monitored.

Cellular IRF3, RXRα, and cytochrome P450 enzyme levels can be determined by measuring the level of protein or mRNA. The levels can be measured using immunoassays such as western blotting, ELISA and the like with an antibody that selectively binds, respectively, to the IRF3, RXTα, or cytochrome P450 enzyme, or a fragment thereof. For measurement of mRNA, amplification, e.g., using PCR, LCR, or hybridization assays, e.g., northern hybridization, RNAse protection, dot blotting, are preferred. The level of protein or mRNA is detected using directly or indirectly labeled detection agents, e.g., fluorescently or radioactively labeled nucleic acids, radioactively or enzymatically labeled antibodies, and the like, as described herein.

Alternatively, IFR3, RXRα, or cytochrome P450 enzyme expression can be measured using a reporter gene system. Such a system can be devised using a corresponding promoter operably linked to a reporter gene such as chloramphenicol acetyltransferase, firefly luciferase, bacterial luciferase, β-galactosidase and alkaline phosphatase. Furthermore, the protein of interest can be used as an indirect reporter via attachment to a second reporter such as red or green fluorescent protein (see, e.g., Mistili & Spector, Nature Biotechnology, 15:961-964 (1997)). The reporter construct is typically transfected into a cell. After treatment with a potential modulator, the amount of reporter gene transcription, translation, or activity is measured according to standard techniques known to those of skill in the art.

### Animal Models

Animal models of IRF3 modulation also find use in screening for modulators of xenobiotic metabolism in health and disease. Similarly, transgenic animal technology including gene knockout technology, for example as a result of homologous recombination with an appropriate gene targeting vector, or gene overexpression, will result in the absence or increased expression of the IRF3. The same technology can also be applied to make knock-out cells. When desired, tissue-specific expression or knockout of the IRF3 protein may be necessary. Transgenic animals generated by such methods find use as animal models of xenobiotic metabolism and are additionally useful in screening for modulators of such metabolism.

Knock-out cells and transgenic mice can be made by insertion of a marker gene or other heterologous gene into an endogenous IRF3 gene site in the mouse genome via homologous recombination. Such mice can also be made by substituting an endogenous IRF3 with a mutated version of the IRF3 gene, or by mutating an endogenous IRF3 gene (e.g., by exposure to carcinogens.)

A DNA construct is introduced into the nuclei of embryonic stem cells. Cells containing the newly engineered genetic lesion are injected into a host mouse embryo, which is re-implanted into a recipient female. Some of these embryos develop into chimeric mice that possess germ cells partially derived from the mutant cell line. Therefore, by breeding the chimeric mice it is possible to obtain a new line of mice containing the introduced genetic lesion (see, e.g., Capecchi et al., Science, 244:1288 (1989)). Chimeric targeted mice can be derived according to Hogan et al., Manipulating the Mouse Embryo: A Laboratory Manual, Cold Spring Harbor Laboratory, (1988); Teratocarcinomas and Embryonic Stem Cells. A Practical Approach, Robertson, ed., IRL Press, Washington, D.C., (1987), and Pinkert, Transgenic Animal Technology: A Laboratory Handbook, Academic Press (2003).

### Screening Methods

Using the assays described herein, one can identify lead compounds that are suitable for further testing to identify those that are therapeutically effective modulating agents by screening a variety of compounds and mixtures of compounds for their ability to decrease, or inhibit the binding of an IRF3 protein to its receptor or to increase the activity or expression of IRF3. Compounds of interest can be either synthetic or naturally occurring.

Screening assays can be carried out in vitro or in vivo. Typically, initial screening assays are carried out in vitro, and can be confirmed in vivo using cell based assays or animal models. Usually a large compound that modulates the activity of IRF3 may be naturally occurring and smaller compounds may be synthetic. The screening methods are designed to screen large chemical libraries by automating the assay steps and providing compounds from any convenient source to assays, which are typically run in parallel (e.g., in microtiter formats on microtiter plates in robotic assays).

The invention provides in vitro assays for identifying modulators of IRF3 activity or expression in a high throughput format. For each of the assay formats described, “no modulator” control reactions which do not include a modulator provide a background level of IRF3 binding interaction to its receptor or receptors. In the high throughput assays of the invention, it is possible to screen up to several thousand different modulators in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (96) modulators. If 1536 well plates are used, then a single plate can easily assay from about 100-about 1500 different compounds. It is possible to assay many different plates per day; assay screens for up to about 6,000-20,000, and even up to about 100,000-1,000,000 different compounds is possible using the integrated systems of the invention. The steps of labeling, addition of reagents, fluid changes, and detection are compatible with full automation, for instance using programmable robotic systems or “integrated systems” commercially available, for example, through BioTX Automation, Conroe, Tex.; Qiagen, Valencia, Calif.; Beckman Coulter, Fullerton, Calif.; and Caliper Life Sciences, Hopkinton, Mass.

Essentially any chemical compound can be tested as a potential inhibitor or modulator of IRF3 activity for use in the methods of the invention. Most preferred are generally compounds that can be dissolved in aqueous or organic (especially DMSO-based) solutions are used. It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland), as well as providers of small organic molecule and peptide libraries ready for screening, including Chembridge Corp. (San Diego, Calif.), Discovery Partners International (San Diego, Calif.), Triad Therapeutics (San Diego, Calif.), Nanosyn (Menlo Park, Calif.), Affymax (Palo Alto, Calif.), ComGenex (South San Francisco, Calif.), and Tripos, Inc. (St. Louis, Mo.).

In one preferred embodiment, modulators of the IRF binding interaction are identified by screening a combinatorial library containing a large number of potential therapeutic compounds (potential modulator compounds). Such “combinatorial chemical or peptide libraries” can be screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res., 37:487-493 (1991) and Houghton et al, Nature, 354:84-88 (1991)). Other chemistries for generating chemical diversity libraries can also be used. Such chemistries include, but are not limited to: peptoids (PCT Publication No. WO 91/19735), encoded peptides (PCT Publication WO 93/20242), random bio-oligomers (PCT Publication No. WO 92/00091), benzodiazepines (U.S. Pat. No. 5,288,514), diversomers such as hydantoins, benzodiazepines and dipeptides (Hobbs et al., Proc. Nat. Acad. Sci. USA, 90:6909-6913 (1993)), vinylogous polypeptides (Hagihara et al., J. Amer. Chem. Soc., 114:6568 (1992)), nonpeptidal peptidomimetics with 1-D-glucose scaffolding (Hirschmann et al, J. Amer. Chem. Soc., 114:9217-9218 (1992)), analogous organic syntheses of small compound libraries (Chen et al., J. Amer. Chem. Soc., 116:2661 (1994)), oligocarbamates (Cho et al., Science, 261:1303 (1993)), and/or peptidyl phosphonates (Campbell et al., J. Org. Chem., 59:658 (1994)), nucleic acid libraries (see, Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem. Tech, Louisville Ky., Symphony, Rainin, Woburn, Mass., 433A Applied Biosystems, Foster City, Calif., 9050 Plus, Millipore, Bedford, Mass.).

siRNA Technology

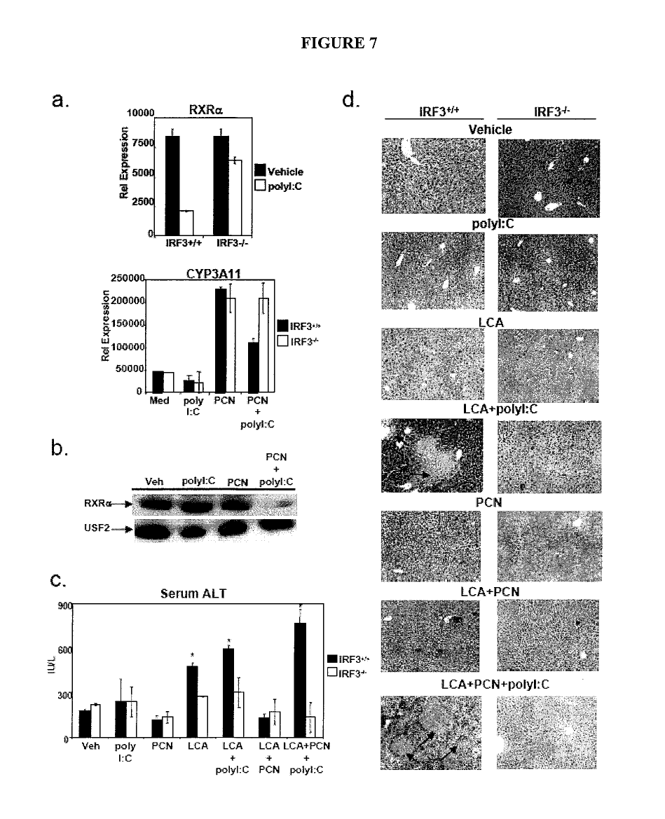

An IRF3 modulator can be an siRNA directed toward inhibiting the expression and tissue levels of IRF3. The design and making of siRNA molecules and vectors are well known to those of ordinary skill in the art. For instance, an efficient process for designing a suitable siRNA is to start at the AUG start codon of the mRNA transcript (see, e.g., FIGS. 7, 8, 9) and scan for AA dinucleotide sequences (see, Elbashir et al., EMBO J. 20: 6877-6888 (2001). Each AA and the 3′ adjacent nucleotides are potential siRNA target sites. The length of the adjacent site sequence will determine the length of the siRNA. For instance, 19 adjacent sites would give a 21 Nucleotide long siRNA siRNAs with 3′ overhanging UU dinucleotides are often the most effective. This approach is also compatible with using RNA pol III to transcribe hairpin siRNAs. RNA pol III terminates transcription at 4-6 nucleotide poly(T) tracts to create RNA molecules having a short poly(U) tail. However, siRNAs with other 3′ terminal dinucleotide overhangs can also effectively induce RNAi and the sequence may be empirically selected. For selectivity, target sequences with more than 16-17 contiguous base pairs of homology to other coding sequences can be avoided by conducting a BLAST search (see, www.ncbi.nln.nih.gov/BLAST).

The siRNA expression vectors to induce RNAi can have different design criteria. A vector can have inserted two inverted repeats separated by a short spacer sequence and ending with a string of T's which serve to terminate transcription. The expressed RNA transcript is predicted to fold into a short hairpin siRNA. The selection of siRNA target sequence, the length of the inverted repeats that encode the stem of a putative hairpin, the order of the inverted repeats, the length and composition of the spacer sequence that encodes the loop of the hairpin, and the presence or absence of 5′-overhangs, can vary. A preferred order of the siRNA expression cassette is sense strand, short spacer, and antisense strand. Hairp siRNAs with these various stem lengths (e.g., 15 to 30) can be suitable. The length of the loops linking sense and antisense strands of the hairpin siRNA can have varying lengths (e.g., 3 to 9 nucleotides, or longer). The vectors may contain promoters and expression enhancers or other regulatory elements which are operably linked to the nucleotide sequence encoding the siRNA. These control elements may be designed to allow the clinician to turn off or on the expression of the gene by adding or controlling external factors to which the regulatory elements are responsive.

### EXAMPLES

The following examples are offered to illustrate, but not to limit the claimed invention.

### Group 1 Examples

### Example 1.1

### Experimental Methods

Specific TLR activation was achieved using polyinosinic:polycytidylic acid (polyI:C) for TLR3 (Amersham Biosciences). Pregnenolone 16alpha-carbonitrile (PCN) and Acetaminophen (ASA) were obtained from Sigma-Aldrich. Ethanol was obtained from Gold Shield Chemical Co.

Age and sex matched 6-9 week old mice were used for all experiments. C57/B16 mice were obtained from Jackson Laboratory. IRF3^{-/-} mice were obtained from Dr. T. Taniguchi. For APAP hepatotoxicity analysis, mice fasted for 36-24 hours and then administered Vehicle (0.1% NaCl) or APAP (175-600 mg/kg) by intraperitoneal injection (i.p.). For serum and histological studies, mice were sacrificed at 6-7 hours post injection and serum and liver samples were retrieved. For survival studies, mice were studied for up to 5 days. For polyI:C studies, mice were also treated with 0.1% NaCl or polyI:C (100 μg) intravenous (i.v.) 12-24 hours prior to APAP treatment. To study the effects of PCN on APAP treatment, mice were treated with PCN (75 mg/kg) or control (1% DMSO, corn oil) by intraperitoneal (i.p.) 12-24 hours prior to APAP treatment. To study the effects of ethanol (EtOH) on APAP treatment, mice were given 20% EtOH in water ad libidum for 5 days prior to APAP therapy. PolyI:C treatment for these experiments occurred at Day 3 and Day 5. Serum alanine aminotransferase (ALT) levels were determined using manufacturer's protocol (TECO Diagnostics). For H&E staining, liver samples were fixed in formalin for 48 hours. H&E stainings were done by UCLA Tissue Procurement Core Laboratory (TPCL).

### RNA Quantitation

For quantitative realtime PCR (Q-PCR), total RNA was isolated and cDNA synthesized according to manufacturer's protocol: Trizol (RNA) and Bio-Rad iScript (cDNA). PCR was then performed using the iCycler thermocycler (Bio-Rad). Q-PCR was conducted in a final volume of 25 μL containing: Taq polymerase, 1×Taq buffer (Stratagene), 125 μM dNTP, SYBR Green I (Molecular Probes), and Fluoroscein (Bio-Rad), using oligo-dT cDNA or random hexamer cDNA as the PCR template. Amplification conditions were: 95° C. (3 min), 40 cycles of 95° C. (20 s), 55° C. (30 s), 72° C. (20 s). Primer sequences are available upon request.

### Example 1.2

### PolyI:C Repression of Key Acetaminophen Metabolizing Genes Depends on IRF3

Cytochrome P450 family members are the first molecules to metabolize acetaminophen (APAP) when it enter the liver. It has been demonstrated that reduced expression or disruption of the signaling processes that regulate cytochrome P450 expression can affect APAP metabolism and hepatotoxicity. Cytochrome P450 family members are critical to APAP hepatotoxicity because they are responsible for the conversion of APAP to its toxic intermediate metabolite, N-acetyl-p-benzoquinoneimine (NAPQI). It is the accumulation of NAPQI that results in cell death and hepatotoxicity. RXRα has previously been shown to regulate key cytochrome P450 family members involved in APAP metabolism, Cyp3A11 and Cyp1A2.

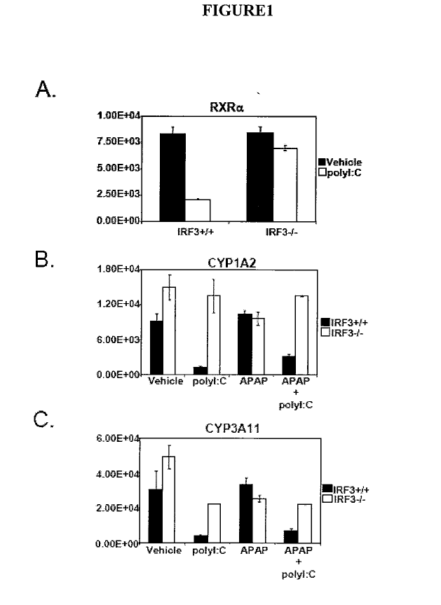

We demonstrated that a single treatment of mice with polyI:C resulted in potent downregulation of RXRα mRNA in an IRF3 dependent manner (FIG. 1A). Previous work has shown that RXRα-deficient hepatocytes have reduced expression. Similar to RXRα-deficient hepatocytes, polyI:C potently repressed basal Cyp1A2 and Cyp3A11 mRNA levels (FIG. 1B,C). It has been suggested that APAP can slightly induce Cyp1A2 and Cyp3A11. Our data, however, indicated that it does not. Furthermore, APAP could not prevent polyI:C from repressing Cyp1A2 and Cyp3A11. PolyI:C is a well known activator of IRF3 and this activation of IRF3 is required for the potent repression of Cyp1A2 and Cyp3A11 (FIG. 1B,C). The fact that activation of IRF3 potently repressed RXRα and cytochrome P450 family members Cyp1A2 and Cyp3A11 suggests that activation of IRF3 can prevent APAP-induced hepatotoxicity, as well as hepatotoxicity that results from the combinatorial treatment of APAP and cytochrome P450 inducing compounds such as PCN and ethanol.

### Example 1.3

### APAP Induction of Serum ALT Levels is Reduced by Treatment with polyI:C

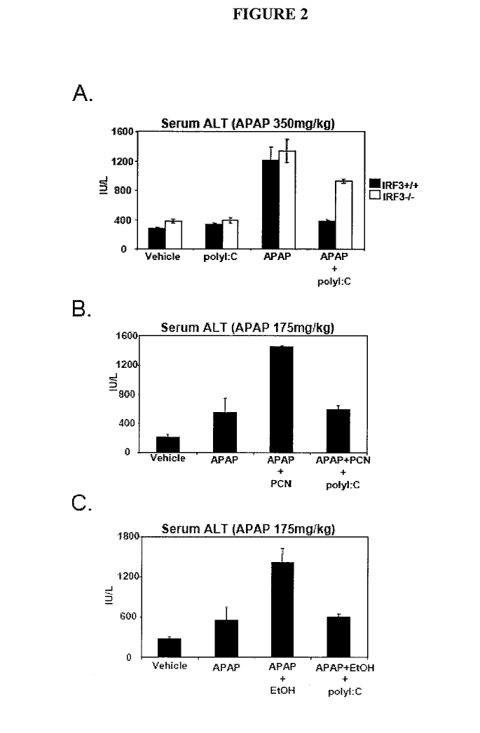

In order to determine if the IRF3 activator, polyI:C, can prevent APAP hepatotoxicity, wildtype and IRF3-deficient mice were treated with APAP and polyI:C. After 6 hrs, mice were sacrificed and analyzed for hepatotoxicity. The hepatic marker enzyme, serum alanine transaminases (ALT), was measured as an indication of hepatotoxicity. As can be seen in FIG. 2A, 350 mg/kg doses of APAP resulted in increased serum ALT levels in both IRF3+/+ and IRF3−/− mice. Pretreatment with polyI:C effectively prevented such increase in serum ALT only when IRF3 was present, demonstrating the requirement of IRF3 in polyI:C prevention of APAP hepatotoxicity.

It has been previously demonstrated that the PXR activator, PCN, can increase APAP hepatotoxicity through induction of cytochrome P450 family members. As demonstrated in FIG. 2B, PCN treatment caused less toxic levels of APAP to result in severe hepatotoxicity as measured by serum ALT. Just as polyI:C was capable of preventing APAP induction of serum ALT levels, polyI:C was capable of preventing PCN/APAP induction of serum ALT levels (FIG. 2B), thus demonstrating that activation of IRF3 was effective at also preventing hepatotoxicity that results from the combination of APAP and cytochrome p450 inducing drugs.

A more common clinical example of hepatotoxicity from APAP and cytochrome P450 inducing substances is APAP therapy following alcohol binging. Regular alcohol intake results in increased cytochrome P450 expression and greater sensitivity to APAP (Dai et al., Exp Mol Pathol 75, 194-200 (2003); McClain et al., Jama 244, 251-253 (1980)). FIG. 2C shows that regular intake of ethanol results in similar sensitivity to APAP as PCN treatment. Furthermore, FIG. 2C shows that polyI:C treatment is effective at preventing ethanol from promoting APAP induction of serum ALT levels.

### Example 1.4

### PolyI:C Prevents Cellular Necrosis from APAP-Induced Hepatotoxicity

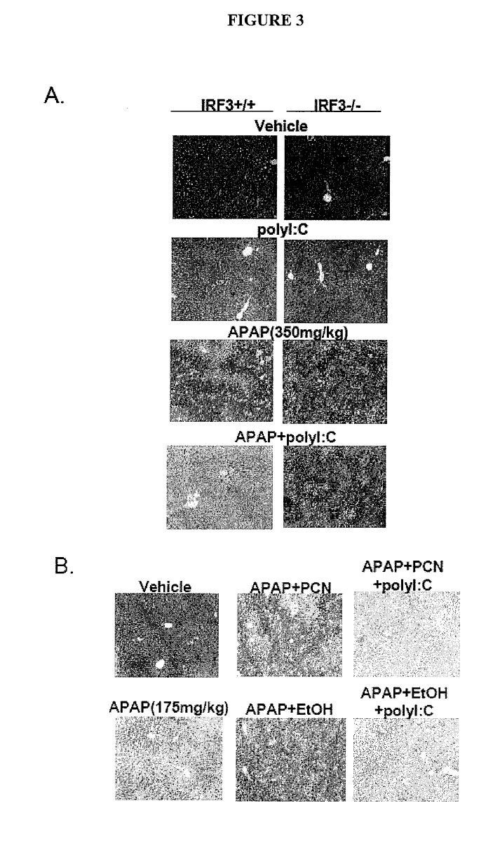

In order to determine if the effects seen in serum ALT levels are truly indicative of the severity of hepatotoxicity, liver sections were analyzed for necrosis and damage by hematoxylin and eosin (H&E) staining. Treatment of mice with 350 mg/kg APAP resulted in severe necrosis by 6 hrs (FIG. 3A). Treatment with polyI:C completely prevented such necrosis from occurring in wildtype mice (FIG. 3A). In mice deficient in IRF3, polyI:C only slightly reduced the severity of necrosis, suggesting that IRF3 plays a significant role in the protection against APAP-induced hepatotoxicity. These results match cytochrome P450 mRNA data, as well as serum ALT data.

Histological analysis of hepatotoxicity was also performed on mice treated with lower levels of APAP in combination with cytochrome P450 inducers, PCN and ethanol. FIG. 3B clearly shows that lower levels of APAP do not exhibit cell necrosis, however, pretreatment with PCN or ethanol results in severe necrosis similar to higher doses of APAP. PolyI:C treatment prevents cell necrosis in these treatments as well. Thus, it is clear that polyI:C treatment is extremely successful at preventing APAP hepatotoxicity and that this process involves IRF3.

### Example 1.5

### Treatment with polyI:C Increases Survival Against APAP-Hepatotoxicity

While our data clearly shows that polyI:C is capable of preventing APAP hepatotoxicity when measured by serum ALT and histological analysis, it is important to determine the effectiveness at preventing death that results from hepatotoxicity and acute liver failure that arises from APAP overdose.

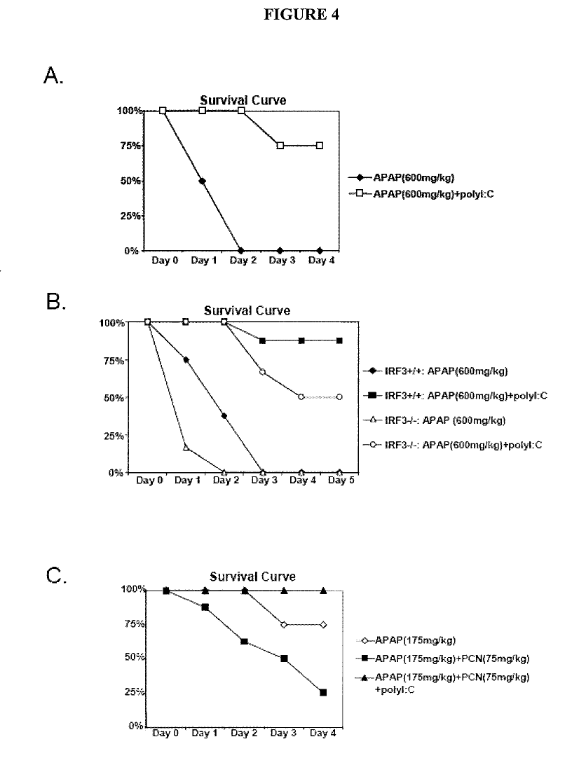

In order to determine the effectiveness of polyI:C in promoting survival against APAP levels that are extremely toxic, mice were treated with 600 mg/kg APAP with or without polyI:C. As demonstrated in FIG. 4A, polyI:C was extremely effective at preventing death from APAP overdose. Interestingly, mice deficient in IRF3 were more sensitive to APAP hepatotoxicity and polyI:C did not protect from APAP overdose in IRF3 deficient mice (FIG. 4B).

It has been previously shown cytochrome P450 inducers such as PCN increase sensitivity to APAP hepatotoxicity and lower the dosage required to cause acute liver failure and overdose. FIG. 4C shows that PCN treatment increases sensitivity to lower levels of APAP and polyI:C treatment prevents overdose from the combination of APAP and cytochrome P450 inducers such as PCN. Thus, polyI:C treatment is extremely effective at preventing death associated with APAP hepatotoxicity, either from excessive APAP or combinatorial intake of cytochrome P450 inducers and lower dosages of APAP.

### Group 2 Examples

### 2.1 Materials and Methods

### 2.1.1 Cell Culture and Mice

Murine bone marrow-derived macrophages (BMMs) were differentiated from marrow as described previously. IFNAR deficient and IRF3 deficient mice were obtained as previously described (Doyle et al., Immunity, 17:251-263 (2002)). Cells from F5 C57B1/6 littermate wild-type mice were used as wild-type controls for experiments using cells from IFNAR^{-/-} and IRF3^{-/-} mice. C57/B16 mice were used for all experiments not involving IFNAR^{-/-} and IRF3^{-/-} mice (Jackson ImmunoResearch Laboratories). RAW 264.7 murine macrophage cells were cultured in DMEM media supplemented with 10% fetal bovine serum and 1% penicillin/streptomycin. Stable Raw-RXRα or Raw-MT vector cells and Huh7-RXRα or Huh7-MT vector cells were made by retroviral transduction and selected with puromycin. Stable Raw-Hes1 or Raw-MT vector was made by transfecting Raw 264.7 cells with 5 μg pCMV-Hes1 or 5 μg pCMV and 0.5 μg pBabe-puro with Superfect (Qiagen) and selected with puromycin.

### 2.1.2 Virus Collection and Quantification

GFP tagged vesicular stomatitis virus was a kind gift from Dr Glen Barber. The virus was grown on a nearly confluent MDCK cells, infected at MOI=0.001. Two days post infection, cell free supernatant was ultra-centrifuged at >100,000 g through a 25% sucrose cushion. The viral pellet was resuspended in PBS. Standard plaque assay was used to determine number of plaque forming units. Briefly, confluent monolayers of MDCK cells in 6 well or 12 well plates were infected in duplicate with serial dilution of the viral stock with intermittent shaking for 1 hour. Subsequently, cells were overlaid with 1×MEM BSA containing 0.7% low melting point agar. Plaques were allowed to develop over 24-36 hours and counted after staining cells with crystal violet.

### 2.1.3 Reagents

Specific PRR activation was achieved using polyinosinic:polycytidylic acid (polyI:C) for TLR3/RIG-I (Amersham Biosciences) and E. coli LPS for TLR4 (Sigma-Aldrich). Synthetic nuclear receptor ligands were obtained as previously described (Castrillo et al., Mol Cell, 12:805-816 (2003)). Lithocholic acid (LCA), pregnenolone 16alpha-carbonitrile (PCN) and acetylsalicylic aid (ASA) were obtained from Sigma-Aldrich. 1,25(OH)₂D₃ (1,25D) was obtained from Biomol. Rifampicin was obtained from Calbiochem. Actinomycin D and Trichostatin A were obtained from Sigma-Aldrich. Macrophage colony-stimulating factor (M-CSF)-containing media was obtained by growing L929 cells 4 days past confluency and then harvesting the conditioned media.

### 2.1.4 Animal Treatments

Age matched 8-10 week old mice were used for all experiments. For hepatic nuclear receptor activation and liver functions analysis, mice were given Vehicle (1% DMSO, corn oil), PCN (75 mg/kg), 1,25D3 (7.5 mg/kg) by gavage and/or LCA (0.25 mg/kg) intraperitoneal (i.p.) for 4 days. For polyI:C studies, mice were also treated with 0.1% NaCl or polyI:C (150 μg) intravenous (i.v.) on Day 1 or Day 3. For viral infection studies, mice were treated with 0.1% NaCl or vesicular stomatitis virus (VSV) (2.5e7 pfu) intravenous (i.v.) on Day 1. On Day 5, mice were sacrificed and serum and liver samples were collected. ASA treatment was done as previously described (Paul et al., Life Sci, 68:457-465 (2000)). ASA treatment was done for 3-4 days. Serum alanine aminotransferase (ALT) (TECO Diagnostics), serum ammonia (Pointe Scientific), blood glucose (LifeScan) and total serum bilirubin (Wako) levels were determined using manufacturer's protocol. P-value determined by t-test (independent) compared to control, unless indicate otherwise. Animal studies were done in accordance with the Animal Research Committee of the University of California, Los Angeles.

### 2.1.5 RNA Quantitation

For quantitative realtime PCR (Q-PCR), total RNA was isolated and cDNA synthesized as described previously. PCR was then performed using the iCycler thermocycler (Bio-Rad). Q-PCR was conducted in a final volume of 25 μL containing: Taq polymerase, 1×Taq buffer (Stratagene), 125 μM dNTP, SYBR Green I (Molecular Probes), and Fluoroscein (Bio-Rad), using oligo-dT cDNA or random hexamer cDNA as the PCR template. Amplification conditions were: 95° C. (3 min), 40 cycles of 95° C. (20 s), 55° C. (30 s), 72° C. (20 s). Primer sequences are available upon request.

### 2.1.7 Western Blot Protein Analysis

For Western blots, cell lysates were incubated at room temperature for 5 min with EB lysis buffer (10 mM Tris.HCl buffer, pH 7.4, containing 5 mM EDTA, 50 mM NaCl, 0.1% (wt/vol) BSA, 1.0% (vol/vol) Triton X-100, protease inhibitors), size-separated in 10% SDS-PAGE, and transferred to nitrocellulose. RXRα and USF2 protein levels were detected using rabbit anti-RXRα or anti-USF2 antibody (Santa Cruz). Whole cell extract from livers were isolated as follows. Livers were briefly homogenized in 1×PBS/protease inhibitors. Homogenized product was centrifuged and pellet was incubated at room temperature for 5 min. with EB buffer.

### 2.1.8 Chromatin Immunoprecipitation

CYP3A4 chromatin immunoprecipitation was done as previously described (Frank et al., J Mol Biol, 346:505-519 (2005)). For RXRα chromatin immunoprecipitation, unactivated and activated cells were fixed at room temperature for 10 min by adding formaldehyde directly to the culture medium to a final concentration of 1%. The reaction was stopped by adding glycine at a final concentration of 0.125 M for 5 min at room temperature. After three ice-cold PBS washes, the cells were collected and lysed for 10 min on ice in cell lysis buffer 5 mM PIPES [piperazine-N,N′-bis(2-ethanesulfonic acid) [pH 8.0], 85 mM KCl, 0.5% NP-40, protease inhibitors. The nuclei were resuspended in nuclei lysis buffer (50 mM Tris-HCl [pH 8.1], 10 mM EDTA, 1% SDS, protease inhibitors) and incubated on ice for 10 min. Chromatin was sheared into 500- to 1,000-bp fragments by sonication and was then precleared with protein A or protein G-Sepharose beads. The purified chromatin was diluted with ChIP dilution buffer (0.01% SDS, 1.1% Triton X-100, 1.2 mM EDTA, 16.7 mM Tris-HCl [pH 8.1], 167 mM NaCl, protease inhibitors) and immunoprecipitated overnight at 4° C. using 2-4 μg of anti-Hes1 (Santa Cruz Biotech) or anti-HDAC1 (Upstate). Immune complexes were collected with protein G-Sepharose beads, washed thoroughly and eluted. After protein-DNA cross-linking was reversed and the DNA was purified, the presence of selected DNA sequences was assessed by PCR PCR products were analyzed on 2% agarose gel and quantified with ImageJ (Rasband, W. S., Image, J., In U.S. National Institutes of Health, Bethesda, Md., USA). Primers used for ChIP are available upon request.

2.1.9 siRNA Assays

Targeted sequence for the Hes1 siRNA duplex or nonspecific siRNA duplex were synthesized by Invitrogen. Duplex oligonucleotides were transfected using Lipofectamine (Invitrogen) at a ratio of 10-20 μmol of RNA to 1.5 μl of Lipofectamine in serum-free, antibiotics-free media. Media was changed after 4-6 hours and experiments were done 36 hours post-transfection. The target sequence for the Hes-1 siRNA was 5′-CGACACCGGACAAACCAAA-3′ (Ross et al., Mol Cell Biol, 24:3505-3513 (2004)). The target sequence for the RXRα siRNA was 5′-AAGCACUAUGGAGUGUACAGC-3′ (Cao et al., Mol Cell Biol, 24:9705-9725 (2004)).

### 2.1.10 Histology

For H&E staining, liver samples were fixed in formalin for 48 hours. H&E stainings were done by UCLA Tissue Procurement Core Laboratory (TPCL). For Oil Red O staining, liver samples were snap frozen in OCT and frozen tissue sections were made by TPCL. Oil Red O staining was done in accordance with manufacturer's protocol (Diagnostic Biosystems). Briefly, slides were placed in Propylene Glycol for 2 min, followed by Oil Red O Staining for 6 min.@60° C. Slides were washed and tissue was differentiated in 85% Propylene Glycol for 1 min, followed by Modified Mayer's Hematoxylin staining for 1 min. Slides were again extensively washed and coverlip was added with an aqueous mounting medium.

### 2.2 Anti-Viral Immune Response Represses RXRα and Liver Metabolism In Vivo

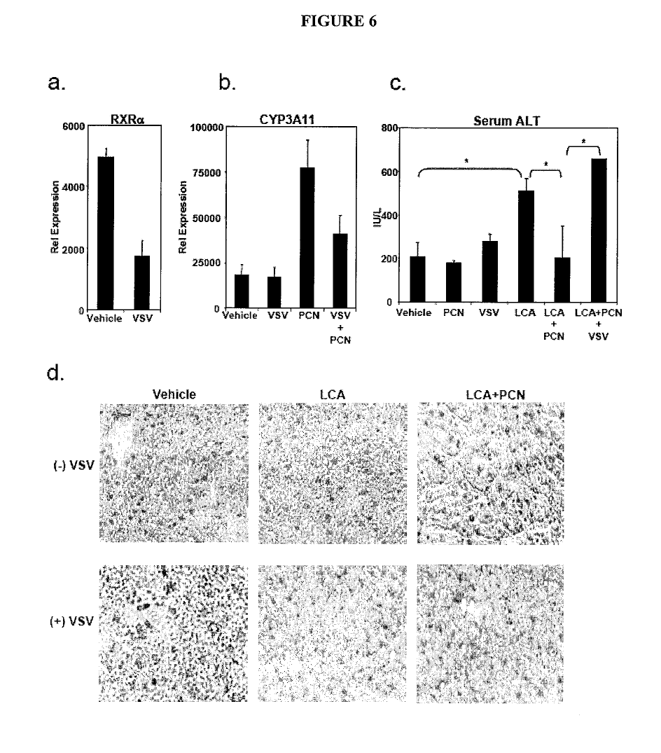

In order to investigate the relationship between liver metabolism and viral infections, C57/B16 mice were infected with vesicular stomatitis virus (VSV) and nuclear receptor function was analyzed. VSV infection potently down regulated expression of RXRα mRNA in vivo (FIG. 6a). Furthermore, down regulation of this critical heterodimeric partner for hepatic nuclear receptors was associated with the inhibition of multiple nuclear receptor pathways, including induction of PXR-mediated CYP3A11 by prenenolone-16alpha-carbonitrile (PCN) and VDR-mediated induction of CYP24 mRNA by 1alpha,25-dihydroxyvitamin D3 (1,25(OH)₂D₃) (FIG. 6b, Supp. 1). Furthermore, VSV infections in the Huh7 hepatocyte cell line resulted in inhibition of hepatic LXR, FXR and PPARα-mediated induction of hepatic nuclear receptor target genes (Supp. 1).

Detoxification and clearance of secondary bile acids, such as lithocholic acid (LCA), is an important metabolic function of the liver required for physiologic homeostasis. Defective metabolism of LCA or excessive amounts of LCA results in cholestasis and hepatotoxicity. PCN activation of PXR/RXR has been previously shown to protect the liver from secondary bile acid (LCA)-induced hepatotoxicity through induction of CYP3A11 and other genes involved in the metabolism of LCA (Xie et al., Proc Natl Acad Sci, 98:3375-3380 (2001); Staudinger et al., Proc Natl Acad Sci USA, 98:3369-3374 (2001)). In wild-type mice, administration of LCA in excess of natural levels led to significant elevation of serum alanine aminotransferase (ALT) levels, which was reduced by co-treatment with PCN (FIG. 6c). In order to determine the impact of viral infection on nuclear receptor-regulated bile acid metabolism, the LCA cholestasis model was analyzed in the context of VSV infection. Although VSV infection alone had no effect on serum ALT levels, it blocked the ability of PCN to reduce LCA-induced serum ALT levels (FIG. 6c). Furthermore, VSV infection induced fatty change and hepatotoxicity in LCA-treated mice, as demonstrated by Oil Red 0 staining (FIG. 6d). The VSV plus LCA-induced hepatotoxicity could not be blocked by the addition of PCN. Thus, viral infections inhibit PXR/RXR-dependent gene expression and promote LCA-induced liver damage.

To determine the mechanism responsible for the inhibition of hepatic gene expression and metabolism observed during viral infection, experiments were repeated with polyinosine-polycytidylic acid (polyI:C), representing viral dsRNA. Treatment with polyI:C resulted in a significant reduction in RXRα mRNA expression (FIG. 7a). Additionally, polyI:C blunted the induction of CYP3A11 by PCN as well as the induction of CYP24 by the VDR agonist 1,25(OH)₂D₃ (FIG. 7a, Supp. 1). Furthermore, hepatic LXR, FXR and PPARα target genes were also inhibited by polyI:C treatment in Huh7 cells (Supp. 1). Both polyI:C and viruses such as VSV are known to activate IRF3, a key mediator of the antiviral immune response. Studies in IRF3 knockout mice established that IRF3 was critical for the repression of RXRα and hepatic nuclear receptor target genes by polyI:C (FIG. 7a, Supp. 1). Furthermore, addition of the nuclear receptor agonist PCN to polyI:C treatment resulted in a further loss of RXRα protein expression (FIG. 7b).

Similar to the results obtained with VSV, treatment of mice with polyI:C alone did not significantly increase serum ALT levels. However, polyI:C in combination with LCA strongly induced liver damage, and this damage was not blocked by PCN (FIG. 7c, d). Moreover, polyI:C failed to promote LCA-mediated increases of serum ALT levels or enhance liver damage in IRF3^{-/-} mice, demonstrating the requirement for IRF3 in polyI:C regulation of hepatic gene expression and function. (FIG. 7c and d). These studies establish that viral activation of IRF3 inhibits hepatic nuclear receptor target gene induction and metabolic activity, resulting in potentiation of LCA-mediated hepatotoxicity.

### Example 2.3

### PolyI:C and LPS Repress RXRα Expression Through IRF3

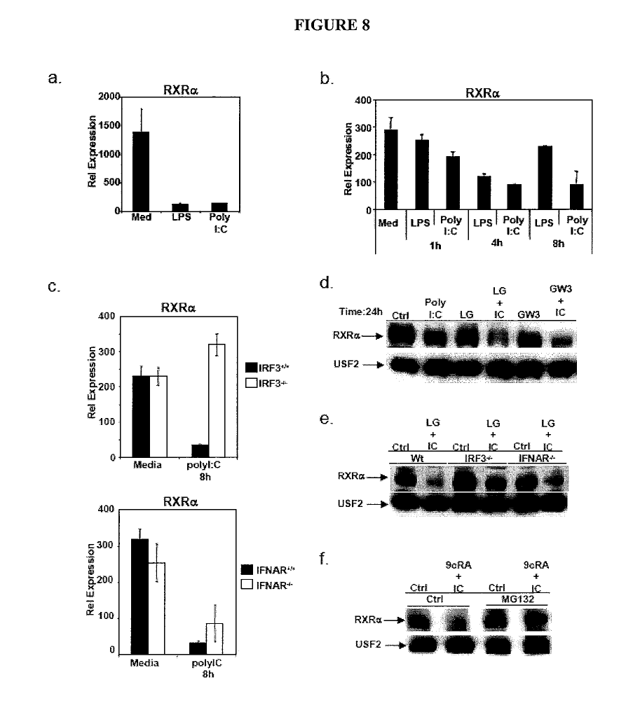

In order to gain a greater understanding of the molecular mechanisms behind innate immune system repression of RXRα and RXRα target genes, we confirmed, by quantitative PCR (Q-PCR), that polyI:C and LPS repressed RXRα mRNA in BMMs (bone marrow derived macrophages) after 4 hours stimulation (FIG. 8a). Furthermore, an extended time course indicated that polyI:C is a more potent repressor of RXRα mRNA than LPS (FIG. 8b). These data validate the in vitro model as representative of our in vivo studies, since RXRα mRNA expression is inhibited by viral infections and TLR ligands in both systems. Protein expression analysis revealed that RXRα protein loss following polyI:C treatment was more obvious upon the addition of RXR-specific (LG268, LG) or LXR-specific (GW3965, GW3) agonists (FIG. 8d). Previously, IRF3 was found to be involved in the repression of LXR target genes in BMMs (Castrillo et al., Mol Cell, 12:805-816 (2003)). Because RXRα cell type specific knockout studies have demonstrated critical roles for RXRα target genes (Sucov et al., Genes Dev, 8:1007-1018 (1994); Imai et al., Proc Natl Acad Sci USA, 98:224-228 (2001); Li et al., Nature, 407:633-636 (2000); Wan et al., Mol Cell Biol, 20:4436-4444 (2000)), we examined the mechanism for such repression in greater detail.

Next, we explored the mechanism of RXRα repression by analyzing the contribution of IRF3 and Type I IFNs, as these are the main signaling mediators shared by TLR3 and TLR4 but not TLR9 in macrophages. PolyI:C-mediated inhibition of RXRα was defective in IRF3^{-/-} BMMs but not IFNAR^{-/-} BMMs (FIG. 8c). Similar regulation was seen at the protein level, as RXRα protein expression levels were significantly higher in IRF3^{-/-} compared to IFNAR^{-/-} BMMs (FIG. 8e). While there was some loss of RXRα protein in IRF3^{-/-} BMMs following polyI:C and LG268 treatment, the protein levels were significantly higher than in WT or IFNAR^{-/-} BMMs, while USF2 levels were equivalent. This data suggests the existence of an IRF3-dependent, Type I IFN-independent pathway for RXRα repression.

Optimal transcription of nuclear receptor target genes is known to require degradation of nuclear receptors, such as RXRα, by the 26S-proteosome complex (Gianni et al., Embo J. 21:3760-3769 (2002)). New protein synthesis replaces degraded nuclear receptors on the promoters of these target genes during transcription (Gianni et al., Embo J, 21:3760-3769 (2002)). We analyzed whether nuclear receptor activation of the 26S-proteosome complex would coordinate with IRF3-mediated inhibition of RXRα mRNA expression to contribute to the loss of RXRα protein. Indeed, MG132, a 26S-proteosome complex inhibitor, prevented loss of RXRα protein following co-stimulation with the RAR/RXR agonist 9-cis retinoic acid (9cRA) and polyI:C in BMMs (FIG. 8f). Thus, maximal RXRα protein loss likely requires combinatorial repression of RXRα mRNA by polyI:C and activation of 26S-proteosome complex mediated degradation by nuclear receptor agonists.

### Example 2.4

### IRF3 Inhibits RXRα Transcription Through Induction of the Transcriptional Suppressor, Hes-1

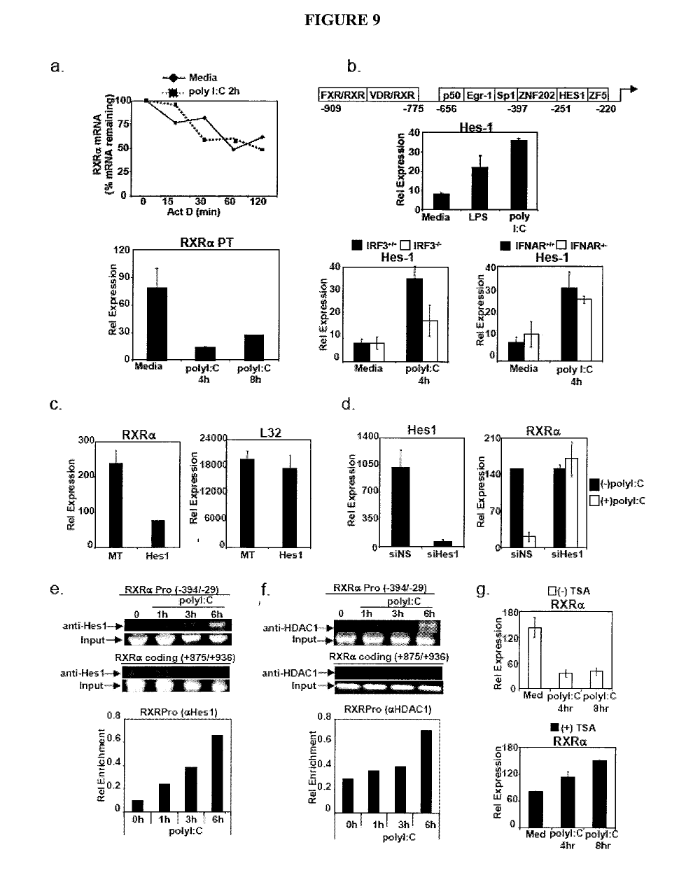

We further analyzed potential transcriptional and post-transcriptional mechanisms through which polyI:C might repress RXRα. BMMs were pretreated with or without polyI:C for 2 hours and then treated with Actinomycin D (a transcription inhibitor) to measure RXRα mRNA stability. No significant differences were observed in RXRα mRNA stability from samples treated with or without polyI:C, suggesting that repression is not post-transcriptionally regulated (FIG. 9a). Furthermore, RXRα primary transcripts measured by Q-PCR using primers that amplify a region spanning an exon and intron were strongly repressed following polyI:C treatment (FIG. 9a). Together, these data indicate that polyI:C regulates RXRα expression at the level of transcription.

In order to gain greater insight into how RXRα is transcriptionally repressed by polyI:C, the promoter region of RXRα (−1 to −1000 bp) was analyzed for predicted transcription factor binding sites. Using promoter analysis software, MatInspector (www.genomatix.de), highly predicted binding sites were identified by core similarity (>0.9) and matrix similarity (>0.9). The first 400 bp of the promoter identified multiple hits for three known transcriptional repressors; Hes1, ZF5 and ZNF202 (FIG. 9b). Hes1 and ZNF202 have previously been identified as potential transcriptional regulators of cholesterol metabolism (Porsch-Ozcurumez et al., J Biol Chem, 276:12427-12433 (2001)); Steffensen et al., Biochem Biophys Res Commun, 312:716-724 (2003)). Hes1 mRNA was potently induced by polyI:C and LPS (FIG. 9b), while ZF5 and ZNF202 mRNA levels were unaffected (data not shown). While it is known that NF-□B activators like TNF-□ can induce Hes1 (Aguilera et al., Proc Natl Acad Sci USA, 101: 16537-16542 (2004)), our data indicate that polyI:C induction of Hes1 also involves IRF3, but not Type I IFNs (FIG. 9b). Preliminary Hes1 promoter analysis indicates an ISRE (−722/−751) with core similarity of 1.0 and matrix similarity of 0.91 (data not shown), but further studies are required to determine if direct binding of IRF3 to the Hes1 promoter is involved in the polyI:C-induced Hes1 upregulation.

To assess the ability of Hes1 to repress RXRα and RXR-related genes, Raw 264.7 cells stably transfected with pCMV-Hes1 were compared to empty vector controls in terms of RXRα mRNA expression and function. FIG. 9c shows that over expression of Hes1 led to the specific down regulation of RXRα mRNA, with control L32 mRNA being unaffected. Furthermore, knockdown experiments with siRNA specific to Hes1 demonstrated the requirement of Hes1 in polyI:C-mediated repression of RXRα (FIG. 9d).

Hes1 mediates gene repression by recruiting the Gro/TLE tetramer and HDAC1 complex to the promoter region of its target genes (Nuthall et al., Mol Cell Biol, 22:389-399 (2002)). Chromatin immunoprecipitation of Hes1 and HDAC1 demonstrated that polyI:C promotes specific recruitment of Hes1 and HDAC1 to the RXRα promoter region and predicted Hes1 binding site (FIG. 9e,f). To test if recruitment of Hes1 and HDAC1 is involved in polyI:C repression of RXRα, BMMs were pretreated with or without the HDAC1 inhibitor, trichostatin A (TSA), followed by stimulation with polyI:C. The addition of TSA prevented polyI:C repression of RXRα, and allowed polyI:C to induce RXRα (FIG. 9g), providing further evidence for a novel mechanism of repression of RXRα by polyI:C.

### Example 2.5

### Transcriptional Repression of RXRα Results in Defective Induction of RXR-Target Genes

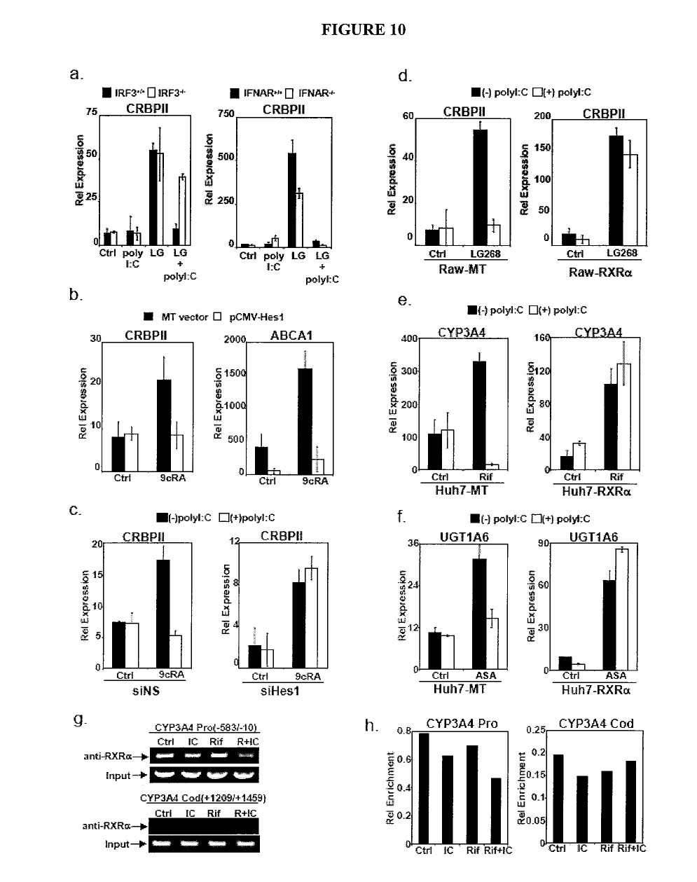

We predicted that the expression of RXRα target genes would mirror regulation of RXRα by polyI:C. Indeed, just as polyI:C induced down regulation of RXRα requires IRF3 and is independent of Type I IFNs, induction of the RXRα target gene CRBPII by synthetic RXR ligand (LG268) was repressed by polyI:C in IFNAR^{-/-} BMMs but not IRF3^{-/-} BMMs (FIG. 10a). Since repression of RXRα by polyI:C appears to require Hes1, we analyzed the role of Hes1 in repression of RXRα target genes. As seen in FIG. 10b, overexpression of Hes1 in RAW 264.7 cells prevents the RAR/RXR agonist, 9cRA, from inducing CRBPII and ABCA1. Furthermore, polyI:C is unable to repress 9cRA induction of CRBPII in cells with knockdown of Hes1 (FIG. 5c).

In order to determine if loss of RXRα contributes to polyI:C repression of nuclear receptor regulated genes, we analyzed RAW 264.7 cells stably expressing RXRα (Supp. 2). PolyI:C was unable to repress LG268 induced CRBPII in the RXRα overexpressing RAW 264.7 cells (FIG. 10d). Additionally, we analyzed if repression of RXRα is a key requirement of polyI:C repression of RXRα target hepatic genes. As seen in FIG. 5e, transfected polyI:C was capable of repressing rifampicin induction of the human homolog to CYP3A11, CYP3A4, in Huh7 cells, a human hepatocyte cell line. In the presence of RXRα overexpression (Supp. 2), however, polyI:C no longer repressed CYP3A4 (FIG. 10e). These results were matched in the induction of another RXR regulated gene, UGT1A6, which is induced by and metabolizes ASA (FIG. 10f) (Ciotti et al., Pharmacogenetics, 7:485-495 (1997); Vyhlidal et al., J Biol Chem, 279:46779-46786 (2004)).

Finally, we also confirmed by chromatin immunoprecipitation that transcriptional repression of RXRα results in a reduction of RXRα present on the promoter of RXRα target hepatic gene, CYP3A4. As shown in FIG. 10g and h, combinatorial treatment of Huh7 cells with rifampicin and polyI:C resulted in maximal loss of RXRα in the PXR/RXR ER6 binding region of CYP3A4, while binding was minimal and unchanged in the upstream coding region. These data present evidence that IRF3-mediated transcriptional repression of RXRα by transfected and non-transfected polyI:C is integral to the repression of RXR-related target genes.

### Example 2.6

### Viral Infection Greatly Enhanced ASA Hepatotoxicity, a Potential Mouse Model of Reye's Syndrome

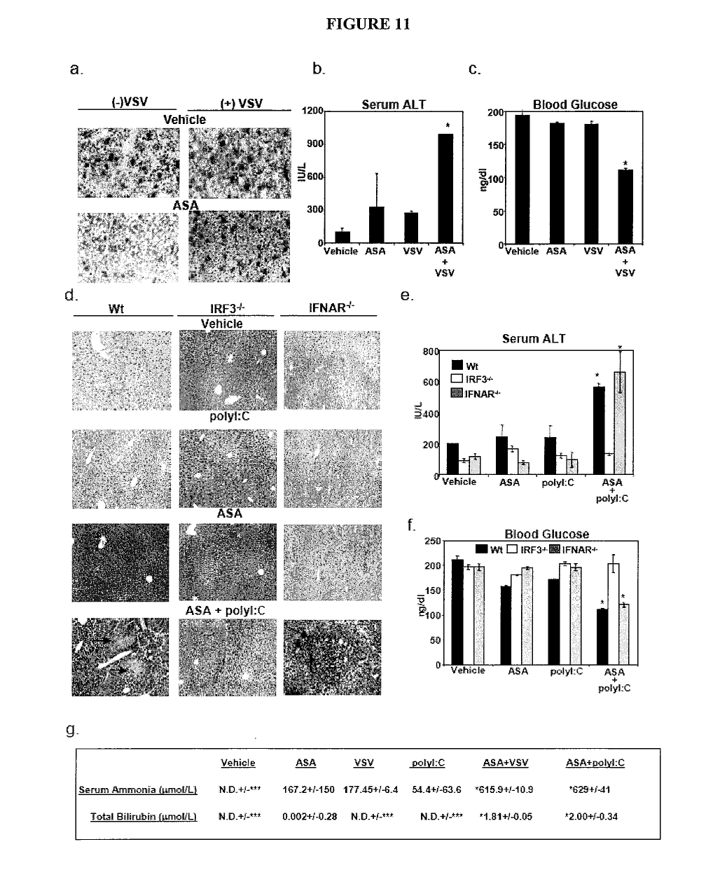

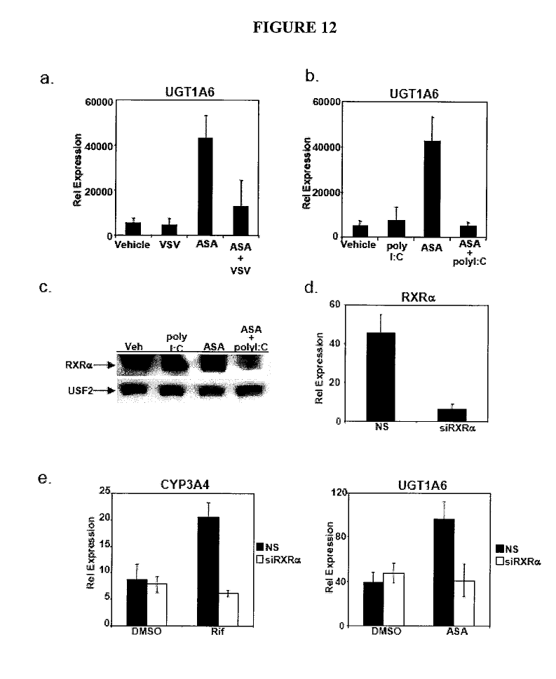

Based on our in vivo and in vitro results, we hypothesized that metabolic disorders involving both nuclear receptor regulated xenobiotic metabolism and viral infections might involve the repression of RXR target genes by IRF3 during host immune response. A human disease that involves viral infection and metabolic hepatotoxicity is Reye's Syndrome, characteristically presenting with delirium and fatty degeneration of the liver in a child with a history of an antecedent viral infection treated with ASA. We speculated that the pathogenesis of Reye's Syndrome might be due, at least in part, to this mechanism of anti-viral immune response and nuclear receptor crosstalk and subsequent metabolic dysfunction. To test this hypothesis, we analyzed the effects of ASA treatment in the presence and absence of an anti-viral immune response initiated by polyI:C or VSV. Treatment of mice with ASA, polyI:C or VSV alone did not cause significant hepatotoxicity. Administration of ASA to mice treated with polyI:C or infected with VSV, however, caused severe hepatotoxicity as evidenced by liver necrosis or fatty degeneration (FIGS. 11a and d). Consistent with a Reye's Syndrome like phenotype, serum ALT, ammonia and total bilirubin levels were increased during co-administration of ASA and polyI:C or VSV, while blood glucose levels were significantly decreased (FIGS. 11b,c,e,f and g) (Belay et al, N Engl J Med 340:1377-1382 (1999); Mitchell et al., Exp Mol Pathol 43:268-273 (1985); Davis et al., Int J Exp Pathol 74:251-258 (1993)). Interestingly, hepatotoxicity from exposure to polyI:C plus ASA did not occur in IRF3^{-/-} mice, but did occur in IFNAR^{-/-} mice (FIG. 11d,e and f). It has been previously shown that polyI:C treatment results in defective ASA metabolism, possibly contributing to the hepatotoxicity seen in our experiment Dolphin et al., Biochem Pharmacol 36:2437-2442 (1987). In addition to CYP3A4 (Dupont et al., Drug Metab Dispos 27:322-326 (1999); Lindell et al., Eur J Clin Invest 33:493-499 (2003)), another enzyme that is induced by ASA and involved in the metabolism of ASA is uridine diphosphate glucuronosyltransferase 1A6 (UGT1A6), whose gene is also regulated by PXR/RXR (Vyhlidal et al., J Biol Chem 279:46779-46786 (2004)). UGT1A6 glucoronidates the ASA intermediate, salicylic acid (Kuehl et al., Drug Metab Dispos 34:199-202 (2006)) and defects in UGT1A6 have been associated with impaired metabolism of aspirin (Ciotti et al., Pharmacogenetics 7:485-495 (1997)). Interestingly, treatment with ASA or the PXR/RXR agonist PCN potently increased UGT1A6 and CYP3A11 mRNA in vivo, but not other PXR/RXR genes such as Oatp2 that are likely not involved in ASA metabolism (FIGS. 6b, 7a, 12a and b, Supp. 4). Furthermore, this induction was diminished by either polyI:C stimulation or VSV infection (FIGS. 6b, 7a, 12a and b, Supp. 4). Additionally, the repression of UGT1A6 by polyI:C was dependent on IRF3 (Supp. 4). The biological loss of RXRα likely contributes to this effect. Just as the loss of RXRα decreases CYP3A11 expression in mice or CYP3A4 in Huh7 cells (FIG. 12e) (Wu et al., Mol Pharmacol 65:550-557 (2004)), UGT1A6 induction by ASA is impaired in Huh7 cells that have RXRα silenced by siRNA (FIG. 12e) and ASA and polyI:C co-treatment resulted in a significant loss of RXRα protein, just as PCN and polyI:C treatment led to the potent loss of RXRα protein (FIG. 12d).

Mechanisms for ASA toxicity are likely through membrane permeability transition (MPT) and mitochondrial injury, which is caused by ASA's intermediate, salicylic acid destabilization of mitochondrial calcium homeostasis (Trost, L. C., and J. J. Lemasters, Toxicol Appl Pharmacol 147:431-441 (1997)). Rhodamine 123 assays demonstrate that RXRα repression by polyI:C results in loss of mitochondrial membrane potential in mock-transfected Huh7 cells co-treated with ASA and polyI:C, but not in Huh7 cells overexpressing RXRα (Supp 2). These in vivo and in vitro observations provide evidence that crosstalk between anti-viral immune responses and nuclear receptor signaling play a critical role in the pathogenesis of Reye's Syndrome.

### REFERENCES

TABLE 1
IRF3 PROTEIN SEQUENCE AND OTHER INFORMATION

IRF3 protein [Homo sapiens] ACCESSION AAH71721
Strausberg, R. L., Feingold, E. A., et al.; Generation and initial
analysis of more than 15,000 full-length human and mouse cDNA
sequences; Proc. Natl. Acad. Sci. U.S.A. 99 (26), 16899-16903 (2002)

SEQ ID NO:1

| | |
|---|---|
| 1 | mgtpkprilp wlvsqldlgq legvawvnks rtrfripwkh glrqdaqqed fgifqawaea |
| 61 | tgayvpgrdk pdlptwkrnf rsalnrkegl rlaedrskdp hdphkiyefv nsgvgdfsqp |
| 121 | dtspdtnggg stsdtqedil dellgnmvla plpdpgppsl avapepcpqp lrspsldnpt |
| 181 | pfpnlgpsen plkrllvpge ewefevtafy rgrqvfqqti scpeglrlvg sevgdrtlpg |
| 241 | wpvtlpdpgm sltdrgvmsy vrhvlsclgg glalwragqw lwaqrlghch tywavseell |
| 301 | pnsghgpdge vpkdkeggvf dlgpfivdli tftegsgrsp ryalwfcvge swpqdqpwtk |
| 361 | rlvmvkvvpt clralvemar vggasslent vdlhisnshp lsltsdqyka ylqdlvegmd |
| 421 | fqgpges |

TABLE 2
IRF3 NUCLEOTIDE SEQUENCE AND OTHER INFORMATION

```
Homo sapiens interferon regulatory factor 3 (IRF3), mRNA, linear.
NM_001571 version NM_001571.2 GI:46403042
REF.1 (bases 1 to 1648), Sankar, S., Chan, H., Romanow, W. J., Li, J.
and Bates, R. J., IKK-i signals through IRF3 and NFkappaB to mediate the
production of inflammatory cytokines, Cell. Signal. 18 (7),
982-993 (2006) - Expression of IKK-i can activate both NFkappaB and
IRF3, leading to the production of several cytokines including
interferon beta.
REF 2 (bases 1 to 1648) Peng, T., Kotla, S., Bumgarner, R. E. and
Gustin, K. E.; Human rhinovirus attenuates the type I interferon
response by disrupting activation of interferon regulatory factor 3; J.
Virol. 80 (10), 5021-5031 (2006) - GeneRIF: Rhinovirus type 14
infection inhibits the host type I interferon in vitro response by
interfering with IRF-3 activation. Erratum:[J Virol. 2006 July;
80(13):6722]
REF 3 (bases 1 to 1648) Loo,Y. M., Owen, D. M., Li, K., Erickson, A. K.,
Johnson, C. L., Fish, P. M., Carney, D. S., Wang, T., Ishida, H.,
Yoneyama, M., Fujita, T., Saito, T., Lee, W. M., Hagedorn, C. H., Lau,
D. T., Weinman, S. A., Lemon, S. M. and Gale, M. Jr.; Viral and
therapeutic control of IFN-beta promoter stimulator 1 during
hepatitis C virus infection, Proc. Natl. Acad. Sci. U.S.A. 103 (15),
6001-6006 (2006) - GeneRIF: HCV infection transiently induces RIG-
I- and IPS-1-dependent IRF-3 activation.
REF 4 (bases 1 to 1648) Korherr, C., Gille, H., Schafer, R.,
Koenig-Hoffmann, K., Dixelius, J., Egland, K. A., Pastan, I. and
Brinkmann, U.; Identification of proangiogenic genes and pathways by
high-throughput functional genomics: TBK1 and the IRF3 pathway; Proc.
Natl. Acad. Sci. U.S.A. 103 (11), 4240-4245 (2006) - GeneRIF: belongs to
one signaling pathway that mediates inductionof gene expression, which,
in concert, mediates proliferative activity toward endothelial cells
REFERENCE (bases 1 to 1648); Zhang, J., Xu, L. G., Han, K. J., Wei, X.
and Shu, H. B.; PIASy represses TRIF-induced ISRE and NF-kappaB
activation but not apoptosis; FEBS Lett. 570 (1-3), 97-101 (2004)
REFERENCE (bases 1 to 1648) Marson, A., Lawn, R. M. and Mikita, T.;
Oxidized low density lipoprotein blocks lipopolysaccharide-induced
interferon beta synthesis in human macrophages by interfering with IRF3
activation; J. Biol. Chem. 279 (27), 28781-28788 (2004) - IRF3
activities are essential for the initiation of transcription of the
IFNbeta gene
Mori, M., Yoneyama, M., Ito, T., Takahashi, K., Inagaki, F. and Fujita,
T.; Identification of Ser-386 of interferon regulatory factor 3
ascritical target for inducible phosphorylation that determines
activation; J. Biol. Chem. 279 (11), 9698-9702 (2004)- GeneRIF: Ser-386
is the target of the IRF-3 kinase and critical determinant for the
activation of IRF-3.
Jiang, Z., Mak, T. W., Sen, G. and Li, X.; Toll-like receptor 3-mediated
activation of NF-kappaB and IRF3 diverges at Toll-IL-1 receptor domain-
containing adapter inducing IFN-beta Proc. Natl. Acad. Sci. U.S.A.
101 (10), 3533-3538 (2004)-double-stranded RNA-induced TLR3/TRIF-
mediated NF-kappaB and IRF3 activation diverge at TRIF
REFERENCE 22 (bases 1 to 1648)
Kim, T. Y., Lee, K. H., Chang, S., Chung, C., Lee, H. W., Yim, J. and
Kim, T. K.; Oncogenic potential of a dominant negative mutant of
interferon regulatory factor 3; J. Biol. Chem. 278 (17), 15272-15278
(2003)- hIRF3 inhibited cell growth, blocked DNA synthesis, and induced
apoptosis, while a dominant negative mutant transformed 3T3 cells,
implying that IRF3 may function as a tumor suppressor and its dominant
negative mutant may have a role in tumorigenesis.
Servant, M. J., Grandvaux, N., tenOever, B. R., Duguay, D., Lin, R. and
Hiscott, J.; Identification of the minimal phosphoacceptor site required
for in vivo activation of interferon regulatory factor 3 in response to
virus and double-stranded RNA, J. Biol. Chem. 278 (11), 9441-9447
(2003) - Ser(396) within the C-terminal Ser/Thr cluster is targeted in
vivo for phosphorylation following virus infection and plays an
essential role in IRF-3 activation
Yang, H., Lin, C. H., Ma, G., Orr, M., Baffi, M. O. and Wathelet, M. G.;
Transcriptional activity of interferon regulatory factor (IRF)-3depends
on multiple protein-protein interactions; Eur. J. Biochem. 269 (24),
6142-6151 (2002) - IRF3 binds to p300/CBP and acts as a transcription
factor.
Peters, K. L., Smith, H. L., Stark, G. R. and Sen, G. C.; IRF-3-
dependent, NFkappa B- and JNK-independent activation of the 561 and
IFN-beta genes in response to double-stranded RNA Proc. Natl. Acad. Sci.
U.S.A. 99 (9), 6322-6327 (2002) - IRF-3-dependent, NFkappa B- and
JNK-independent activation of the 561 and IFN-beta genes in response to
double-stranded RNA
/translation=
“MGTPKPRILPWLVSQLDLGQLEGVAWVNKSRTRFRIPWKHGLRQ
DAQQEDFGIFQAWAEATGAYVPGRDKPDLPTWKRNFRSALNRKEGLRLAEDRSKDPHD
PHKIYEFVNSGVGDFSQPDTSPDTNGGGSTSDTQEDILDELLGNMVLAPLPDPGPPSL
AVAPEPCPQPLRSPSLDNPTPFPNLGPSENPLKRLLVPGEEWEFEVTAFYRGRQVFQQ
TISCPEGLRLVGSEVGDRTLPGWPVTLPDPGMSLTDRGVMSYVRHVLSCLGGGLALWR
AGQWLWAQRLGHCHTYWAVSEELLPNSGHGPDGEVPKDKEGGVFDLGPFIVDLITFTE
GSGRSPRYALWFCVGESWPQDQPWTKRLVMVKVVPTCLRALVEMARVGGASSLENTVD
                LHISNSHPLSLTSDQYKAYLQDLVEGMDFQGPGES”
    variation 533
                        /gene=“IRF3”
                        /replace=“a”
                        /replace=“g”
    variation 1013
                        /gene=“IRF3”
                        /replace=“a”
                        /replace=“g”
    variation 1375
                        /gene=“IRF3”
                        /replace=“a”
                        /replace=“g”
    STS 1453..1602
                        /gene=“IRF3”
                        /standard_name=“NIB1805”
                        /db_xref=“UniSTS:12987”
    STS 1455..1589
                        /gene=“IRF3”
                        /standard_name=“G62110”
                        /db_xref=“UniSTS:139152”
    variation 1455
                        /gene=“IRF3”
                        /replace=“c”
                        /replace=“t”
    variation 1526
                        /gene=“IRF3”
                        /replace=“c”
                        /replace=“g”
    polyA_signal 1585..1590
                        /gene=“IRF3”
ORIGIN
```

SEQ ID NO:2

cDNA

| | |
|---|---|
| 1 | cgtagaacca gataggggcg ggaacagccc agcgggccgt cccatcggct tttgggtctg |
| 61 | ttacccaaag aatgataaag ttggttttat ttcaagaagt cgatcgaaaa gaaagcccca |
| 121 | gcgctctaga gctcagctga cgggaaaggg ggtgcgcagc ctcgagtttg agagctaccc |
| 181 | ggagccccaa gacagggggg ggttccagct gcccgcacgc cccgaccttc catcgtaggc |
| 241 | cggaccatgg gaaccccaaa gccacggatc ctgccctggc tggtgtcgca gctggacctg |
| 301 | gggcaactgg agggcgtggc ctgggtgaac aagagccgca cgcgcttccg catcccttgg |
| 361 | aagcacggcc tacggcagga tgcacagcag gaggatttcg gaatcttcca ggcctgggcc |
| 421 | gaggccactg gtgcatatgt tcccgggagg gataagccag acctgccaac ctggaagagg |
| 481 | aatttccgct ctgccctcaa ccgcaaagaa gggttgcgtt tagcagagga ccggagcaag |
| 541 | gaccctcacg acccacataa aatctacgag tttgtgaact caggagttgg ggacttttcc |
| 601 | cagccagaca cctctccgga caccaatggt ggaggcagta cttctgatac ccaggaagac |
| 661 | attctggatg agttactggg taacatggtg ttggccccac tcccagatcc gggaccccca |
| 721 | agcctggctg tagcccctga gccctgccct cagcccctgc ggagccccag cttggacaat |
| 781 | cccactccct tcccaaacct ggggccctct gagaacccac tgaagcggct gttggtgccg |
| 841 | ggggaagagt gggagttcga ggtgacagcc ttctaccggg gccgccaagt cttccagcag |
| 901 | accatctcct gcccggaggg cctgcggctg gtggggtccg aagtgggaga caggacgctg |
| 961 | cctggatggc cagtcacact gccagaccct ggcatgtccc tgacagacag gggagtgatg |
| 1021 | agctacgtga ggcatgtgct gagctgcctg ggtgggggac tggctctctg gcgggccggg |
| 1081 | cagtggctct gggcccagcg gctggggcac tgccacacat actgggcagt gagcgaggag |
| 1141 | ctgctcccca acagcgggca tgggcctgat ggcgaggtcc ccaaggacaa ggaaggaggc |
| 1201 | gtgtttgacc tggggccctt cattgtagat ctgattacct tcacggaagg aagcggacgc |
| 1261 | tcaccacgct atgccctctg gttctgtgtg ggggagtcat ggccccagga ccagccgtgg |
| 1321 | accaagaggc tcgtgatggt caaggttgtg cccacgtgcc tcagggcctt ggtagaaatg |
| 1381 | gcccgggtag ggggtgcctc ctccctggag aatactgtgg acctgcacat ttccaacagc |
| 1441 | cacccactct ccctcacctc cgaccagtac aaggcctacc tgcaggactt ggtggagggc |
| 1501 | atggatttcc agggccctgg ggagagctga gccctcgctc ctcatggtgt gcctccaacc |
| 1561 | cccctgttcc ccaccacctc aaccaataaa ctggttcctg ctatgaaaaa aaaaaaaaaa |
| 1621 | aaaaaaaaaa aaaaaaaaaa aaaaaaaa |

Each publication, patent application, patent, and other reference cited herein is incorporated by reference in its entirety to the extent that it is not inconsistent with the present disclosure. In particular, all publications cited herein are incorporated herein by reference in their entirety for the purpose of describing and disclosing the methodologies, reagents, and tools, and biological activities of IRF3 reported in the publications that can be used in the methods, modulators, and compositions of the invention. Nothing herein is to be construed as an admission that the invention is not entitled to antedate such disclosure by virtue of prior invention.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A method of treating a subject for exposure to a compound capable of being metabolized by action of a tissue Cytochrome P450 enzyme to form a metabolite of the compound wherein the metabolite is toxic to the tissue, said method comprising administering to said subject in need thereof an effective amount of a modulator of IRF3.

2. A method of claim 1, wherein the tissue is lung tissue, liver tissue, or kidney tissue.

3. A method of claim 1, wherein the compound is acetaminophen.

4. A method of claim 3, wherein the acetaminophen is co-administered with the modulator.

5. A method of claim 3, wherein the modulator is administered after the acetaminophen.

6. A method of claim 5, wherein the patient is suspected of having or has ingested an overdose of acetaminophen.

7. A method of claim 1, wherein the compound is a halogenated compound.

8. A method of claim 1, wherein the enzyme comprises Cytochrome P450 3A11 and Cytochrome P450 1A2.

9. A method of claim 1, wherein the enzyme comprises a Cytochrome P450 isoform selected from Cytochrome P450 1A2, Cytochrome P450 2B6, Cytochrome P450 2C19, Cytochrome P450 2C9, Cytochrome P450 2D6, Cytochrome P450 2E1, and Cytochrome P450 3A 4, 5, or 7.

10. A method of claim 1, wherein the compound is a procarcinogen and the metabolite is a carcinogen.

11. A method of claim 1, wherein the compound is a medicinal agent co-formulated with the modulator.

12. A method of claim 1, wherein the metabolite is a reactive intermediate capable of covalently reacting with tissue macromolecules.

13. A method of claim 1, wherein the metabolite is a free radical or can become converted to a free radical.

14. A method of claim 1, wherein the subject was exposed to an inducer of the Cytochrome P450 enzyme.

15. A method of modulating the metabolism or effects of a compound by Cytochrome P450 enzyme system in a subject exposed to a compound, said method comprising administering an effective amount of the modulator to a patient before, during or after the exposure to the compound.

16. A method of claim 15, wherein the major route of the metabolism or disposition or effect of the compound in the subject is by the Cytochrome P450 enzyme system.

17. A method of claim 15, wherein the metabolism mediates a toxicity of the compound.

18. A method of claim 15, wherein the compound is a drug and the exposure is by administration of the drug to the subject.

19. A method of claim 18, wherein the compound is acetaminophen.

20. (canceled)

21. (canceled)

22. A method of preventing or reducing the induction of a Cytochrome P450 enzyme in a subject exposed to a compound capable of inducing the enzyme, said method comprising administering an effective amount of the modulator to the subject.

23. A method of claim 22, wherein the subject is human.

24. A method of any of the above claims claim 1 wherein the IRF3 modulator is polyI:C, polyC:G, dsRNA, R848, LPS, a Toll-receptor modulator, or a TRIF modulator.

25. A pharmaceutical composition comprising a first agent which is a drug which is a substrate for a Cytochrome 450 enzyme system and a second agent which is an IRF3 modulator.

26. A composition of claim 25, wherein the drug is acetaminophen.

27. A composition of claim 25, wherein the drug is metabolized to a toxic compound by the action of the Cytochrome 450 enzyme system.

28. A pharmaceutical composition comprising a first agent which induces a Cytochrome P450 enzyme and a second agent which is an IRF3 modulator.

29. A pharmaceutical composition comprising N-acetyl cysteine and an IRF3 modulator.

30. A pharmaceutical composition of claim 25, wherein the modulator is polyI:C, polyC:G, dsRNA, R848, LPS, a Toll-receptor modulator, or a TRIF modulator.

31. A method of modulating the expression, activity, or levels of a cytochrome P450 enzyme, said method comprising administering to a subject a modulator of IRF3 or a modulator of any of the members of a Cytochrome P450 enzyme expression control pathway of FIG. 5 or 13 having IRF3 as a member.
